(19) United States
(12) Patent Application Publication
(10) Pub. No.: US 2010/0222322 A1
(43) Pub. Date: Sep. 2, 2010

(54) Non-Nucleoside Reverse Transcriptase Inhibitors

(75) Inventors: Scott E. Wolkenberg, Jenkintown, PA (US); Zhijian Zhao, Wilmington, PA (US); Craig W. Lindsley, Schwenksville, PA (US)

Correspondence Address: MERCK, P O BOX 2000, RAHWAY, NJ, 07065-0907, US

(73) Assignee: MERCK & CO., INC., Rahway, NJ (US)

(21) Appl. No.: 11/922,682

(22) PCT Filed: Jun. 23, 2006
(86) PCT No.: PCT/US2006/024434

§ 371 Date: Dec. 20, 2007

Related U.S. Application Data
(60) Provisional application No. 60/694,600, filed on Jun. 28, 2005.
(60) Provisional application No. 60/707,364, filed on Aug. 11, 2005.

(51) Int. Cl. A61K 31/404; A61P 31/18; C07D 403/12; C07D 401/12; A61K 31/454; A61K 31/496

(52) U.S. Cl. 514/210.21; 514/414; 548/467; 546/201; 514/323; 514/254.09; 544/373

(57) ABSTRACT

Indole compounds of Formula (I) are HIV reverse transcriptase inhibitors, wherein R¹, R², R³, R⁴ and R⁵ are defined herein. The compounds of Formula (I) and their pharmaceutically acceptable salts are useful in the inhibition of HIV reverse transcriptase, the prophylaxis and treatment of infection by HIV and in the prophylaxis, delay in the onset, and treatment of AIDS. The compounds and their salts can be employed as ingredients in pharmaceutical compositions, optionally in combination with other antivirals, immunomodulators, antibiotics or vaccines.

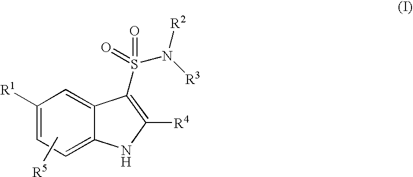

### FIELD OF THE INVENTION

The present invention is directed to the use of certain indoles and their pharmaceutically acceptable salts for the inhibition of HIV reverse transcriptase, the prophylaxis and treatment of HIV infection and HIV replication, and the prophylaxis, delay in the onset of and treatment of AIDS.

### BACKGROUND OF THE INVENTION

The retrovirus designated human immunodeficiency virus (HIV), particularly the strains known as HIV type-1 (HIV-1) and type-2 (HIV-2) viruses, have been etiologically linked to the immunosuppressive disease known as acquired immunodeficiency syndrome (AIDS). HIV seropositive individuals are initially asymptomatic but typically develop AIDS related complex (ARC) followed by AIDS. Affected individuals exhibit severe immunosuppression which makes them highly susceptible to debilitating and ultimately fatal opportunistic infections. Replication of HIV by a host cell requires integration of the viral genome into the host cell's DNA. Since HIV is a retrovirus, the HIV replication cycle requires transcription of the viral RNA genome into DNA via an enzyme know as reverse transcriptase (RT).

Reverse transcriptase has three known enzymatic functions: The enzyme acts as an RNA-dependent DNA polymerase, as a ribonuclease, and as a DNA-dependent DNA polymerase. In its role as an RNA-dependent DNA polymerase, RT transcribes a single-stranded DNA copy of the viral RNA. As a ribonuclease, RT destroys the original viral RNA and frees the DNA just produced from the original RNA. And as a DNA-dependent DNA polymerase, RT makes a second, complementary DNA strand using the first DNA strand as a template. The two strands form double-stranded DNA, which is integrated into the host cell's genome by the integrase enzyme.

It is known that compounds that inhibit enzymatic functions of HIV RT will inhibit HIV replication in infected cells. These compounds are useful in the prophylaxis or treatment of HIV infection in humans. Among the compounds approved for use in treating HIV infection and AIDS are the RT inhibitors 3′-azido-3′-deoxythymidine (AZT), 2′,3′-dideoxyinosine (ddI), 2′,3′-dideoxycytidine (ddC), d4T, 3TC, nevirapine, delavirdine, efavirenz and abacavir.

While each of the foregoing drugs is effective in treating HIV infection and AIDS, there remains a need to develop additional HIV antiviral drugs including additional RT inhibitors. A particular problem is the development of mutant HIV strains that are resistant to the known inhibitors. The use of RT inhibitors to treat AIDS often leads to viruses that are less sensitive to the inhibitors. This resistance is typically the result of mutations that occur in the reverse transcriptase segment of the pol gene. The continued use of antiviral compounds to prevent HIV infection will inevitably result in the emergence of new resistant strains of HIV. Accordingly, there is a particular need for new RT inhibitors that are effective against mutant HIV strains.

The following references are of interest as background:

WO94/19321 and EP530907 each disclose certain indole compounds as HIV reverse transcriptase inhibitors useful in the prevention or treatment of HIV infection and the treatment of AIDS.

GB 2,282,808 discloses certain 3-substituted heterocyclic indoles as inhibitors of HIV reverse transcriptase and its resistant varieties.

WO 02/083216 A1 and WO 2004/014364 A1 each disclose certain substituted phenylindoles for the treatment of HIV.

WO2004/014300 and WO2004/014851 each disclose certain indole compounds as tyrosine kinase inhibitors, wherein the compounds have certain acyl groups in the 2-position and certain sulfonyl groups in the 3-position of the indole ring.

Williams et al., J. Med. Chem. 1993, vol. 36, pp. 1291-1294 discloses 5-chloro-3-(phenylsulfonyl)indole-2-carboxamide as a non-nucleoside inhibitor of HIV-1 reverse transcriptase.

Young et al., Bioorg. & Med. Chem. Letters 1995, vol. 5, pp. 491-496 discloses certain 2-heterocyclic indole-3-sulfones as inhibitors of HIV-1 reverse transcriptase.

### SUMMARY OF THE INVENTION

The present invention is directed to indole compounds and their use in the inhibition of HIV reverse transcriptase, the prophylaxis of infection by HIV, the treatment of infection by REV, and the prophylaxis, treatment, and delay in the onset of AIDS and/or ARC. More particularly, the present invention includes a method for the inhibition of HIV reverse transcriptase, the treatment or prophylaxis of HIV infection, or the treatment or prophylaxis or delay in the onset of AIDS, wherein the method comprises administering to a subject in need thereof an effective amount of a compound of Formula I, or a pharmaceutically acceptable salt thereof:

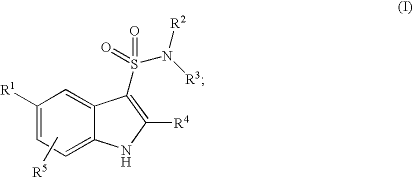

wherein:

### R¹ is:

(1) halogen,
(2) CN,
(3) NO₂,
(4) C(O)R^{A},
(5) C(O)OR^{A},
(6) C(O)N(R^{A})R^{B},
(7) SR^{A},
(8) S(O)R^{A},
(9) S(O)₂R^{A},
(10) S(O)₂N(R^{A})R^{B},
(11) N(R^{A})R^{B},
(12) N(R^{A})S(O)₂R^{B},
(13) N(R^{A})C(O)R^{B},
(14) N(R^{A})C(O)ORB,
(15) N(R^{A})S(O)₂N(R^{A})R^{B},
(16) OC(O)N(R^{A})R^{B},
(17) N(R^{A})C(O)N(R^{A})R^{B},
(18) C₁₋₆ alkyl,
(19) C₁₋₆ haloalkyl,
(20) C₂₋₆ alkenyl,
(21) C₂₋₆ alkynyl,
(22) OH,
(23) O—C₁₋₆ alkyl,
(24) O—C₁₋₆ haloalkyl,
(25) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(26) CycA,
(27) AryA,
(28) HetA,
(29) HetR,
(30) C₁₋₆ alkyl substituted with CycA, AryA, HetA, or HetR,
(31) J-CycA,
(32) J-AryA,
(33) J-HetA, or
(34) J-HetR;
   J is O, S, S(O), S(O)₂, O—C₁₋₆ alkylene, S—C₁₋₆ alkylene, S(O)—C₁₋₆ alkylene, S(O)₂—C₁₋₆ alkylene, N(R^{A}), N(R^{A})—C₁₋₆ alkylene, C(O), C(O)—C₁₋₆ alkylene-O, C(O)N(R^{A}), C(O)N(R^{A})—C₁₋₆ alkylene, C(O)N(R^{A})—C₁₋₆ alkylene-C(O)O, or C(O)N(R^{A})S(O)₂;

### R² is:

(1) H,
(2) C₁₋₆ alkyl,
(3) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B}, with the proviso that the OH, O—C₁₋₆ alkyl, or O—C₁₋₆ haloalkyl is not attached to the carbon in C₁₋₆ alkyl that is directly attached to the rest of the molecule,
(4) O—C₁₋₆ alkyl,
(5) CycB,
(6) AryB,
(7) HetB,
(8) HetS, or
(9) C₁₋₆ alkyl substituted with CycB, AryB, HetB, or HetS;

### R³ is:

(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B}, with the proviso that the OH, O—C₁₋₆ alkyl, or O—C₁₋₆ haloalkyl is not attached to the carbon in C₁₋₆ alkyl that is directly attached to the rest of the molecule,
(3) CycB,
(4) AryB,
(5) HetB,
(6) HetS, or
(7) C₁₋₆ alkyl substituted with CycB, AryB, HetB, or HetS;
   alternatively R² and R³ together with the N atom to which they are attached form a 4- to 7-membered, saturated or mono-unsaturated heterocyclic ring or a 6- to 10-membered saturated or mono-unsaturated, bridged or fused heterobicyclic ring, wherein the heterocyclic or heterobicyclic ring optionally contains a heteroatom in addition to the nitrogen attached to R² and R³ selected from N, O, and S, wherein the S is optionally oxidized to S(O) or S(O)₂, and wherein the heterocyclic or heterobicyclic ring is optionally substituted with a total of from 1 to 4 substituents, wherein:
(i) from zero to 4 substituents are each independently halogen, CN, C₁₋₆ alkyl, OH, oxo, O—C₁₋₆ alkyl, C₁₋₆ haloalkyl, O—C₁₋₆ haloalkyl, C(O)R^{A}, C(O)OR^{A}, S(O)₂R-A, C₁₋₆ alkylene-CN, C₁₋₆ alkylene-OH, or C₁₋₆ alkylene-O—C₁₋₆ alkyl, and
(ii) from zero to 1 substituent is CycB, AryB, HetB, or C₁₋₆ alkyl substituted with CycB, AryB, or HetB;

### R⁴ is:

(1) C(O)OH,
(2) C(O)OR^{U},
(3) C(O)NH₂, or
(4) C(O)NR^{V}R^{W};
   R⁵ is H or independently has the same definition as R¹;

### R^{U} is:

(1) C₁₋₆ alkyl, or
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B};
   R^{V} is H or C₁₋₆ alkyl;

### R^{W} is:

(1) H,
(2) C₁₋₆ alkyl,
(3) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B}, with the proviso that the OH, O—C₁₋₆ alkyl, or O—C₁₋₆ haloalkyl is not attached to the carbon in C₁₋₆ alkyl that is directly attached to the rest of the molecule,
(4) CycC,
(5) AryC,
(6) HetC,
(7) HetT, or
(8) C₁₋₆ alkyl substituted with CycC, AryC, HetC, or HetT;
   CycA is C₃₋₈ cycloalkyl which is optionally substituted with a total of from 1 to 6 substituents, wherein:

(i) from zero to 6 substituents are each independently:
(1) halogen,
(2) CN,
(3) C₁₋₆ alkyl,
(4) OH,
(5) O—C₁₋₆ alkyl, or
(6) C₁₋₆ haloalkyl, and

(ii) from zero to 2 substituents are each independently:
(1) CycD,
(2) AryD,
(3) HetD, or
(4) C₁₋₆ alkyl substituted with AryD, HetD, or CycD;
   AryA is aryl which is optionally substituted with a total of from 1 to 6 substituents, wherein:

(i) from zero to 6 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) halogen,
(8) CN,
(9) NO₂,
(10) N(R^{A})R^{B},
(11) C(O)N(R^{A})R^{B},
(12) C(O)R^{A},
(13) C(O)—C₁₋₆ haloalkyl,
(14) C(O)OR^{A},
(15) OC(O)N(R^{A})R^{B},
(16) SR^{A},
(17) S(O)R^{A},
(18) S(O)₂R^{A},
(19) S(O)₂N(R^{A})R^{B},
(20) N(R^{A})S(O)₂R^{B},
(21) N(R^{A})S(O)₂N(R^{A})R^{B},
(22) N(R^{A})C(O)R^{B},
(23) N(R^{A})C(O)N(R^{A})R^{B},
(24) N(R^{A})C(O)—C(O)N(R^{A})R^{B}, or
(25) N(R^{A})CO₂R^{B}, and

(ii) from zero to 2 substituents are each independently:
(1) CycD,
(2) AryD,
(3) HetD, or
(4) C₁₋₆ alkyl substituted with AryD, HetD, or CycD;
   HetA is heteroaryl which is optionally substituted with a total of from 1 to 6 substituents, wherein:

(i) from zero to 6 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) oxo,
(8) halogen,
(9) CN,
(10) NO₂,
(11) N(R^{A})R^{B},
(12) C(O)N(R^{A})R^{B},
(13) C(O)R^{A},
(14) C(O)—C₁₋₆ haloalkyl,
(15) C(O)OR^{A},
(16) OC(O)N(R^{A})R^{B},
(17) SR^{A},
(18) S(O)R^{A},
(19) S(O)₂R^{A},
(20) S(O)₂N(R^{A})R^{B},
(21) N(R^{A})S(O)₂R^{B},
(22) N(R^{A})S(O)₂N(R^{A})R^{B},
(23) N(R^{A})C(O)R^{B},
(24) N(R^{A})C(O)N(R^{A})R^{B},
(25) N(R^{A})C(O)—C(O)N(R^{A})R^{B}, or
(26) N(R^{A})CO₂R^{B}, and

(ii) from zero to 2 substituents are each independently:
(1) CycD,
(2) AryD,
(3) HetD, or
(4) C₁₋₆ alkyl substituted with AryD, HetD, or CycD;
   each CycB independently has the same definition as CycA;
   each AryB independently has the same definition as AryA;
   each HetB independently has the same definition as HetA;
   CycC independently has the same definition as CycA;
   AryC independently has the same definition as AryA;
   HetC independently has the same definition as HetA;
   each CycD is independently C₃₋₈ cycloalkyl which is optionally substituted with from 1 to 4 substituents each of which is independently halogen, CN, C₁₋₆ alkyl, OH, O—C₁₋₆ alkyl, C₁₋₆ haloalkyl, C₁₋₆ alkylene-CN, C₁₋₆ alkylene-OH, or C₁₋₆ alkylene-O—C₁₋₆ alkyl;
   each AryD is independently phenyl or naphthyl, wherein the phenyl or naphthyl is optionally substituted with from 1 to 5 substituents each of which is independently halogen, CN, NO₂, C₁₋₆ alkyl, C₁₋₆ haloalkyl, OH, O—C₁₋₆ alkyl, O—C₁₋₆ haloalkyl, N(R^{A})R^{B}, C(O)N(R^{A})R^{B}, C(O)R^{A}, C(O)OR^{A}, SR^{A}, S(O)R^{A}, S(O)₂R^{A}, S(O)₂N(R^{A})R^{B}, S(O)₂N(R^{A})C(O)R^{B}, C₁₋₆ alkylene-CN, C₁₋₆ alkylene-NO₂, C₁₋₆ alkylene-OH, C₁₋₆ alkylene-O—C₁₋₆ alkyl, C₁₋₆ alkylene-O—C₁₋₆ haloalkyl, C₁₋₆ alkylene-N(R^{A})R^{B}, C₁₋₆ alkylene-C(O)N(R^{A})R^{B}, C₁₋₆ alkylene-C(O)R^{A}, C₁₋₆ alkylene-C(O)OR^{A}, C₁₋₆ alkylene-SR^{A}, C₁₋₆ alkylene-S(O)R^{A}, C₁₋₆ alkylene-S(O)₂R^{A}, C₁₋₆ alkylene-S(O)₂N(R^{A})R^{B}, or C₁₋₆ alkylene-S(O)₂N(R^{A})C(O)R^{B};
   each HetD is independently a 5- or 6-membered heteroaromatic ring containing from 1 to 4 heteroatoms independently selected from N, O and S, wherein each N is optionally in the form of an oxide, and wherein the heteroaromatic ring is optionally substituted with from 1 to 4 substituents each of which is independently halogen, CN, NO₂, C₁₋₆ alkyl, C₁₋₆ haloalkyl, OH, O—C₁₋₆ alkyl, O—C₁₋₆ haloalkyl, N(R^{A})R^{B}, C(O)N(R^{A})R^{B}, C(O)R^{A}, C(O)OR^{A}, SR^{A}, S(O)R^{A}, S(O)₂R^{A}, S(O)₂N(R^{A})R^{B}, S(O)₂N(R^{A})C(O)R^{B}, C₁₋₆ alkylene-CN, C₁₋₆ alkylene-NO₂, C₁₋₆ alkylene-OH, C₁₋₆ alkylene-O—C₁₋₆ alkyl, C₁₋₆ alkylene-O—C₁₋₆ haloalkyl, C₁₋₆ alkylene-N(R^{A})R^{B}, C₁₋₆ alkylene-C(O)N(R^{A})R¹³, C₁₋₆ alkylene-C(O)R^{A}, C₁₋₆ alkylene-C(O)OR^{A}, C₁₋₆ alkylene-SR^{A}, C₁₋₆ alkylene-S(O)R^{A}, C₁₋₆ alkylene-S(O)₂R^{A}, C₁₋₆ alkylene-S(O)₂N(R^{A})R^{B}, or C₁₋₆ alkylene-S(O)₂N(R^{A})C(O)R^{B};
   HetR is a 4- to 7-membered, saturated or mono-unsaturated heterocyclic ring containing at least one carbon atom and from 1 to 4 heteroatoms independently selected from N, O and S, where the S is optionally oxidized to S(O) or S(O)₂, and wherein the saturated or mono-unsaturated heterocyclic ring is optionally substituted with from 1 to 4 substituents each of which is independently halogen, CN, C₁₋₆ alkyl, OH, oxo, O—C₁₋₆ alkyl, C₁₋₆ haloalkyl, C(O)R^{A}, C(O)OR^{A}, S(O)₂R^{A}, C₁₋₆ alkylene-CN, C₁₋₆ alkylene-OH, or C₁₋₆ alkylene-O—C₁₋₆ alkyl;
   each HetS independently has the same definition as HetR;
   HetT independently has the same definition as HetR;
   each aryl is independently (i) phenyl, (ii) a 9- or 10-membered bicyclic, fused carbocylic ring system in which at least one ring is aromatic, or (iii) an 11- to 14-membered tricyclic, fused carbocyclic ring system in which at least one ring is aromatic;
   each heteroaryl is independently (i) a 5- or 6-membered heteroaromatic ring containing from 1 to 4 heteroatoms independently selected from N, O and S, wherein each N is optionally in the form of an oxide, or (ii) a 9- or 10-membered bicyclic, fused ring system containing from 1 to 4 heteroatoms independently selected from N, O and S, wherein either one or both of the rings contain one or more of the heteroatoms, at least one ring is aromatic, each N is optionally in the form of an oxide, and each S in a ring which is not aromatic is optionally S(O) or S(O)₂;
   each R^{A} is independently H or C₁₋₆ alkyl; and
   each R^{B} is independently H or C₁₋₆ alkyl.

Embodiments, aspects and features of the present invention are either further described in or will be apparent from the ensuing description, examples and appended claims.

### DETAILED DESCRIPTION OF THE INVENTION

The compounds of Formula I, and pharmaceutically acceptable salts thereof, are HIV reverse transcriptase inhibitors. The compounds are useful for inhibiting HIV reverse transcriptase and for inhibiting HIV replication in vitro and in vivo. More particularly, the compounds of Formula I inhibit the polymerase function of HIV-1 reverse transcriptase. Based upon the testing of representative compounds of Formula I in the assay set forth in Example 70 below, it is known that compounds of Formula I inhibit the RNA-dependent DNA polymerase activity of HIV-1 reverse transcriptase. The compounds can also exhibit activity against drug resistant forms of HIV (e.g., mutant strains of HIV in which reverse transcriptase has a mutation at lysine 103→asparagine (K103N) and/or tyrosine 181→cysteine (Y181C)), and thus can exhibit decreased cross-resistance against currently approved antiviral therapies.

Accordingly, a first embodiment of the present invention is a method as originally set forth above (i.e., as defined and described in the Summary of the Invention), wherein the HIV reverse transcriptase being inhibited is a mutant form of a wild-type reverse transcriptase, and the HIV infection being treated or prevented and the AIDS being treated or prevented or delayed is due to a mutant strain of HIV containing a mutant form of HIV reverse transcriptase. In an aspect of this embodiment, the reverse transcriptase in the mutant strain of HIV has either or both of the K103N and Y181C mutations.

A second embodiment of the present invention is a method as originally set forth above or as set forth in the first embodiment, wherein in the compound of Formula I, or a pharmaceutically acceptable salt thereof, all the variables are as originally defined (i.e., as defined in the Summary of the Invention); and with the proviso that when R⁵ is H, then R¹ is:
(1) C(O)R^{A},
(2) C(O)OR^{A},
(3) C(O)N(R^{A})R^{B},
(4) SR^{A},
(5) S(O)R^{A},
(6) S(O)₂R^{A},
(7) S(O)₂N(R^{A})R^{B},
(8) N(C₁₋₆ alkyl)S(O)₂R^{B},
(9) N(C₁₋₆ alkyl)C(O)R^{B},
(10) N(R^{A})C(O)ORB,
(11) N(R^{A})S(O)₂N(R^{A})R^{B},
(12) OC(O)N(R^{A})R^{B},
(13) N(R^{A})C(O)N(R^{A})R^{B},
(14) C₁₋₆ alkyl,
(15) C₁₋₆ haloalkyl,
(16) C₂₋₆ alkenyl,
(17) C₂₋₆ alkynyl,
(18) OH,
(19) O—C₁₋₆ alkyl,
(20) O—C₁₋₆ haloalkyl,
(21) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(22) CycA,
(23) AryA,
(24) HetA,
(25) HetR,
(26) C₁₋₆ alkyl substituted with CycB, AryB, HetB, or HetR,
(27) J-CycA,
(28) J-AryA,
(29) J-HetA, or
(30) J-HetR.

A third embodiment of the present invention is a method as originally set forth above or as set forth in the first embodiment, wherein in the compound of Formula I, or a pharmaceutically acceptable salt thereof, all the variables are as originally defined; and with the proviso that when R⁵ is H, then R¹ is:
(1) C(O)R^{A},
(2) C(O)OR^{A},
(3) C(O)N(R^{A})R^{B},
(4) SR^{A},
(5) S(O)R^{A},
(6) S(O)₂R^{A},
(7) S(O)₂N(R^{A})R^{B},
(8) N(R^{A})C(O)ORB,
(9) N(R^{A})S(O)₂N(R^{A})R^{B},
(10) OC(O)N(R^{A})R^{B},
(11) N(R^{A})C(O)N(R^{A})R^{B},
(12) C₁₋₆ alkyl,
(13) C₁₋₆ haloalkyl,
(14) C₂₋₆ alkenyl,
(15) C₂₋₆ alkynyl,
(16) OH,
(17) O—C₁₋₆ alkyl,
(18) O—C₁₋₆ haloalkyl,
(19) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(20) CycA,
(21) AryA,
(22) HetA,
(23) HetR,
(24) C₁₋₆ alkyl substituted with CycB, AryB, HetB, or HetR,
(25) J-CycA,
(26) J-AryA,
(27) J-HetA, or
(28) J-HetR.

A fourth embodiment of the present invention is a method as originally set forth above or as set forth in the first embodiment, wherein in the compound of Formula I, or a pharmaceutically acceptable salt thereof, R¹ is:
(1) F, Cl, or Br,
(2) CN,
(3) NO₂,
(4) C(O)—C₁₋₄ alkyl,
(5) C(O)O—C₁₋₄ alkyl,
(6) C(O)N(R^{A})R^{B},
(7) S—C₁₋₄ alkyl,
(8) S(O)—C₁₋₄ alkyl,
(9) S(O)₂—C₁₋₄ alkyl,
(10) S(O)₂N(R^{A})R^{B},
(11) N(R^{A})R^{B},
(12) N(H)S(O)₂—C₁₋₄ alkyl,
(13) N(H)C(O)—C₁₋₄ alkyl,
(14) N(C₁₋₄ alkyl)S(O)₂—C₁₋₄ alkyl,
(15) N(C₁₋₄ alkyl)C(O)—C₁₋₄ alkyl,
(16) N(H)C(O)O—C₁₋₄ alkyl,
(17) N(C₁₋₄ alkyl)C(O)O—C₁₋₄ alkyl,
(18) N(H)S(O)₂N(R^{A})R^{B},
(19) N(C₁₋₄ alkyl)S(O)₂N(R^{A})R^{B},
(20) OC(O)N(R^{A})R^{B},
(21) N(H)C(O)N(R^{A})R^{B},
(22) N(C₁₋₄ alkyl)C(O)N(R^{A})R^{B},
(23) C₁₋₄ alkyl,
(24) C₁₋₄ fluoroalkyl,
(25) C₂₋₄ alkenyl,
(26) C₂₋₄ alkynyl,
(27) OH,
(28) O—C₁₋₄ alkyl,
(29) O—C₁₋₄ fluoroalkyl,
(30) C₁₋₄ alkyl substituted with OH, O—C₁₋₄ alkyl, O—C₁₋₄ fluoroalkyl, CN, NO₂, N(R^{A})R^{B}, C(O)N(R^{A})R^{B}, C(O)—C₁₋₄ alkyl, CO₂—C₁₋₄ alkyl, S—C₁₋₄ alkyl, S(O)—C₁₋₄ alkyl, S(O)₂—C₁₋₄ alkyl, S(O)₂N(R^{A})R^{B}, N(H)C(O)—C₁₋₄ alkyl, N(C₁₋₄ alkyl)C(O)—C₁₋₄ alkyl, N(H)CO₂—C₁₋₄ alkyl, N(C₁₋₄ allyl)CO₂—C₁₋₄ alkyl, N(H)S(O)₂—C₁₋₄ alkyl, N(C₁₋₄ alkyl)S(O)₂—C₁₋₄ alkyl, N(H)S(O)₂N(R^{A})R^{B}, N(C₁₋₄ alkyl)S(O)₂N(R^{A})R^{B}, OC(O)N(R^{A})R^{B}, N(H)C(O)N(R^{A})R^{B}, or N(C₁₋₄ alkyl)C(O)N(R^{A})R^{B},
(31) CycA,
(32) AryA,
(33) HetA,
(34) HetR, or
(35) C₁₋₄ alkyl substituted with CycA, AryA, HetA, or HetR;
   R⁵ is H or independently has the same definition as R¹;
   and all other variables are as originally defined.

A fifth embodiment of the present invention is a method as originally set forth above or as set forth in the first embodiment, wherein in the compound of Formula I, or a pharmaceutically acceptable salt thereof, R² is:
(1) C₁₋₄ alkyl,
(2) C₁₋₄ alkyl substituted with OH, O—C₁₋₄ alkyl, O—C₁₋₄ fluoroalkyl, CN, NO₂, N(R^{A})R^{B}, C(O)N(R^{A})R^{B}, C(O)—C₁₋₄ alkyl, CO₂—C₁₋₄ alkyl, S—C₁₋₄ alkyl, S(O)—C₁₋₄ alkyl, S(O)₂—C₁₋₄ alkyl, S(O)₂N(R^{A})R^{B}, N(H)C(O)—C₁₋₄ alkyl, N(C₁₋₄ alkyl)C(O)—C₁₋₄ alkyl, N(H)CO₂—C₁₋₄ alkyl, N(C₁₋₄ alkyl)CO₂—C₁₋₄ alkyl, N(H)S(O)₂—C₁₋₄ alkyl, N(C₁₋₄ alkyl)S(O)₂—C₁₋₄ alkyl, N(H)S(O)₂N(R^{A})R^{B}, N(C₁₋₄ alkyl)S(O)₂N(R^{A})R^{B}, OC(O)N(R^{A})R^{B}, N(H)C(O)N(R^{A})R^{B}, or N(C₁₋₄ alkyl)C(O)N(R^{A})R^{B}, with the proviso that the OH, O—C₁₋₄ alkyl, or O—C₁₋₄ haloalkyl is not attached to the carbon in C₁₋₄ alkyl that is directly attached to the rest of the molecule,
(3) O—C₁₋₄ alkyl,
(4) CycB,
(5) AryB,
(6) HetB,
(7) HetS, or
(8) C₁₋₄ alkyl substituted with CycB, AryB, HetB, or HetS;
   R³ is H or C₁₋₄ alkyl;
   alternatively R² and R³ together with the N atom to which they are attached form a 4- to 7-membered, saturated or mono-unsaturated heterocyclic ring or a 6- to 10-membered saturated or mono-unsaturated, bridged or fused heterobicyclic ring, wherein the heterocyclic or heterobicyclic ring optionally contains a heteroatom in addition to the nitrogen attached to R² and R³ selected from N, O, and S, wherein the S is optionally oxidized to S(O) or S(O)₂, and wherein the heterocyclic or heterobicyclic ring is optionally substituted with from 1 to 4 substituents each of which is independently Cl, Br, F, CN, C₁₋₄ alkyl, OH, oxo, O—C₁₋₄ alkyl, C₁₋₄ fluoroalkyl, O—C₁₋₄ fluoroalkyl, C(O)—C₁₋₄ alkyl, C(O)O—C₁₋₄ alkyl, S(O)₂—C₁₋₄ alkyl, C₁₋₄ alkylene-CN, C₁₋₄ alkylene-OH, or Cl₁₋₄ alkylene-O—C₁₋₄ alkyl;
   and all other variables are as originally defined.

A sixth embodiment of the present invention is a method as originally set forth above or as set forth in the first embodiment, wherein in the compound of Formula I, or a pharmaceutically acceptable salt thereof, R⁴ is: (1) C(O)O—C₁₋₄ alkyl, (2) C(O)NH₂, or (3) C(O)NR^{V}R^{W}; R^{V} is H or C₁₋₄ alkyl; and R^{W} is:
(1) C₁₋₄ alkyl,
(2) C₁₋₄ alkyl substituted with OH, O—C₁₋₄ alkyl, CN, N(R^{A})R^{B}, C(O)N(R^{A})R^{B}, C(O)—C₁₋₄ alkyl, CO₂—C₁₋₄ alkyl, S—C₁₋₄ alkyl, S(O)—C₁₋₄ alkyl, S(O)₂—C₁₋₄ alkyl, S(O)₂N(R^{A})R^{B}, N(H)C(O)—C₁₋₄ alkyl, N(C₁₋₄ allyl)C(O)—C₁₋₄ alkyl, N(H)S(O)₂—C₁₋₄ alkyl, or N(C₁₋₄ alkyl)S(O)₂—C₁₋₄ alkyl, with the proviso that the OH or O—C₁₋₄ alkyl is not attached to the carbon in C₁₋₄ alkyl that is directly attached to the rest of the molecule,
(3) CycC,
(4) AryC,
(5) HetC,
(6) HetT, or
(7) C₁₋₄ alkyl substituted with CycC, AryC, HetC, or HetT;
   and all other variables are as originally defined.

A seventh embodiment of the present invention is a method as originally set forth above or as set forth in the first embodiment, wherein in the compound of Formula I, or a pharmaceutically acceptable salt thereof:

CycA is C₃₋₆ cycloalkyl which is optionally substituted with a total of from 1 to 4 substituents, wherein:

(i) from zero to 4 substituents are each independently:
(1) Cl, Br, or F,
(2) CN,
(3) C₁₋₄ alkyl,
(4) OH,
(5) O—C₁₋₄ alkyl, or
(6) C₁₋₄ fluoroalkyl, and

(ii) from zero to 1 substituent which is:
(1) CycD,
(2) AryD,
(3) HetD, or
(4) C₁₋₄ alkyl substituted with AryD, HetD, or CycD;
   AryA is phenyl or naphthyl, wherein the phenyl or naphthyl is optionally substituted with a total of from 1 to 5 substituents, wherein:

(i) from zero to 5 substituents are each independently:
(1) C₁₋₄ alkyl,
(2) C₁₋₄ alkyl substituted with OH, O—C₁₋₄ alkyl, O—C₁₋₄ fluoroalkyl, CN, NO₂, N(R^{A})R^{B}, C(O)N(R^{A})R^{B}, C(O)—C₁₋₄ alkyl, CO₂—C₁₋₄ alkyl, S—C₁₋₄ alkyl, S(O)—C₁₋₄ alkyl, S(O)₂—C₁₋₄ alkyl, S(O)₂N(R^{A})R^{B}, N(H)C(O)—C₁₋₄ alkyl, N(C₁₋₄ alkyl)C(O)—C₁₋₄ alkyl, N(H)CO₂—C₁₋₄ alkyl, N(C₁₋₄ alkyl)CO₂—C₁₋₄ alkyl, N(H)S(O)₂—C₁₋₄ alkyl, N(C₁₋₄ alkyl)S(O)₂—C₁₋₄ alkyl, N(H)S(O)₂N(R^{A})R^{B}, N(C₁₋₄ alkyl)S(O)₂N(R^{A})R^{B}, OC(O)N(R^{A})R^{B}, N(H)C(O)N(R^{A})R^{B}, or N(C₁₋₄ alkyl)C(O)N(R^{A})R^{B};
(3) O—C₁₋₄ alkyl,
(4) C₁₋₄ fluoroalkyl,
(5) O—C₁₋₄ fluoroalkyl,
(6) OH,
(7) Cl, Br, or F,
(8) CN,
(9) NO₂,
(10) N(R^{A})R^{B},
(11) C(O)N(R^{A})R^{B},
(12) C(O)—C₁₋₄ alkyl,
(13) C(O)—C₁₋₄ fluoroalkyl,
(14) C(O)O—C₁₋₄ alkyl,
(15) OC(O)N(R^{A})R^{B},
(16) S—C₁₋₄ alkyl,
(17) S(O)—C₁₋₄ alkyl,
(18) S(O)₂—C₁₋₄ alkyl,
(19) S(O)₂N(R^{A})R^{B},
(20) N(H)S(O)₂—C₁₋₄ alkyl,
(21) N(C₁₋₄ alkyl)S(O)₂—C₁₋₄ alkyl,
(22) N(H)C(O)—C₁₋₄ alkyl,
(23) N(C₁₋₄ alkyl)C(O)—C₁₋₄ alkyl,
(24) N(H)CO₂—C₁₋₄ alkyl, or
(25) N(C₁₋₄ allyl)CO₂—C₁₋₄ alkyl, and

(ii) from zero to 1 substituent which is:
(1) CycD,
(2) AryD,
(3) HetD, or
(4) C₁₋₄ alkyl substituted with AryD, HetD, or CycD;
   HetA is (i) a 5- or 6-membered heteroaromatic ring containing from 1 to 4 heteroatoms independently selected from N, O and S, wherein each N is optionally in the form of an oxide, or (ii) a 9- or 10-membered bicyclic, fused ring system containing a total of from 1 to 4 heteroatoms independently selected from zero to 4 N atoms, zero to 2 O atoms, and zero to 2 S atoms, wherein either one or both of the rings contain one or more of the heteroatoms, at least one ring is aromatic, each N is optionally in the form of an oxide, and each S in a ring which is not aromatic is optionally S(O) or S(O)₂; wherein the heteroaromatic ring or the bicyclic, fused ring system is optionally substituted with a total of from 1 to 4 substituents, wherein:

(i) from zero to 4 substituents are each independently:
(1) C₁₋₄ alkyl,
(2) C₁₋₄ alkyl substituted with OH, O—C₁₋₄ alkyl, O—C₁₋₄ fluoroalkyl, CN, NO₂, N(R^{A})R^{B}, C(O)N(R^{A})R^{B}, C(O)—C₁₋₄ alkyl, CO₂—C₁₋₄ alkyl, S—C₁₋₄ alkyl, S(O)—C₁₋₄ alkyl, S(O)₂—C₁₋₄ alkyl, S(O)₂N(R^{A})R^{B}, N(H)C(O)—C₁₋₄ alkyl, N(C₁₋₄ alkyl)C(O)—C₁₋₄ alkyl, N(H)CO₂—C₁₋₄ alkyl, N(C₁₋₄ allyl)CO₂—C₁₋₄ alkyl, N(H)S(O)₂—C₁₋₄ alkyl, N(C₁₋₄ alkyl)S(O)₂—C₁₋₄ alkyl, N(H)S(O)₂N(R^{A})R^{B}, N(C₁₋₄ alkyl)S(O)₂N(R^{A})R^{B}, OC(O)N(R^{A})R^{B}, N(H)C(O)N(R^{A})R^{B}, or N(C₁₋₄ allyl)C(O)N(R^{A})R^{B};
(3) O—C₁₋₄ alkyl,
(4) C₁₋₄ fluoroalkyl,
(5) O—C₁₋₄ fluoroalkyl,
(6) OH,
(7) oxo.
(8) Cl, Br, or F,
(9) CN,
(10) NO₂,
(11) N(R^{A})R^{B},
(12) C(O)N(R^{A})R^{B},
(13) C(O)—C₁₋₄ alkyl,
(14) C(O)—C₁₋₄ fluoroalkyl,
(15) C(O)O—C₁₋₄ alkyl,
(16) OC(O)N(R^{A})R^{B},
(17) S—C₁₋₄ alkyl,
(18) S(O)—C₁₋₄ alkyl,
(19) S(O)₂—C₁₋₄ alkyl,
(20) S(O)₂N(R^{A})R^{B},
(21) N(H)S(O)₂—C₁₋₄ alkyl,
(22) N(C₁₋₄ alkyl)S(O)₂—C₁₋₄ alkyl,
(23) N(H)C(O)—C₁₋₄ alkyl,
(24) N(C₁₋₄ alkyl)C(O)—C₁₋₄ alkyl,
(25) N(H)CO₂—C₁₋₄ alkyl, or
(26) N(C₁₋₄ alkyl)CO₂—C₁₋₄ alkyl, and

(ii) from zero to 1 substituent which is:
(1) CycD,
(2) AryD,
(3) HetD, or
(4) C₁₋₄ alkyl substituted with AryD, HetD, or CycD;
   CycB independently has the same definition as CycA;
   AryB independently has the same definition as AryA;
   HetB independently has the same definition as HetA;
   CycC independently has the same definition as CycA;
   AryC independently has the same definition as AryA;
   HetC independently has the same definition as HetA;
   each CycD is independently C₃₋₆ cycloalkyl which is optionally substituted with from 1 to 4 substituents each of which is independently Cl, Br, F, C₁₋₄ alkyl, OH, O—C₁₋₄ alkyl, C₁₋₄ fluoroalkyl, C₁₋₄ alkylene-OH, or C₁₋₄ alkylene-O—C₁₋₄ alkyl;
   each AryD is independently phenyl, wherein the phenyl is optionally substituted with from 1 to 5 substituents each of which is independently Cl, Br, F, CN, NO₂, C₁₋₄ alkyl, C₁₋₄ fluoroalkyl, OH, O—C₁₋₄ alkyl, O—C₁₋₄ fluoroalkyl, N(R^{A})R^{B}, C(O)N(R^{A})R^{B}, C(O)—C₁₋₄ alkyl, C(O)O—C₁₋₄ alkyl, S—C₁₋₄ alkyl, S(O)—C₁₋₄ alkyl, S(O)₂—C₁₋₄ alkyl, S(O)₂N(R^{A})R^{B}, S(O)₂N(R^{A})C(O)—C₁₋₄ alkyl, C₁₋₄ alkylene-OH, C₁₋₄ alkylene-O—C₁₋₄ alkyl, C₁₋₄ alkylene-N(R^{A})R^{B}, C₁₋₄ alkylene-C(O)N(R^{A})R^{B}, or C₁₋₄ alkylene-S(O)₂N(R^{A})R^{B};
   each HetD is independently a 5- or 6-membered heteroaromatic ring containing from 1 to 4 heteroatoms independently selected from N, O and S, wherein each N is optionally in the form of an oxide, and wherein the heteroaromatic ring is optionally substituted with from 1 to 4 substituents each of which is independently Cl, Br, F, CN, NO₂, C₁₋₄ alkyl, C₁₋₄ fluoroalkyl, OH, O—C₁₋₄ alkyl, or O—C₁₋₄ fluoroalkyl;
   each HetR independently is a 4- to 7-membered, saturated or mono-unsaturated heterocyclic ring containing at least one carbon atom and from 1 to 4 heteroatoms independently selected from N, O and S, where the S is optionally oxidized to S(O) or S(O)₂, and wherein the saturated or mono-unsaturated heterocyclic ring is optionally substituted with from 1 to 4 substituents each of which is independently

Cl, Br, F, C₁₋₄ alkyl, oxo, O—C₁₋₄ alkyl, C₁₋₄ fluoroalkyl, C(O)—C₁₋₄ alkyl, C(O)O—C₁₋₄ alkyl, S(O)₂—C₁₋₄ alkyl, or C₁₋₄ alkylene-O—C₁₋₄ alkyl;

HetS independently has the same definition as HetR; and
HetT independently has the same definition as HetR;
and all other variables are as originally defined or as defined in the second, third, fourth, fifth or sixth embodiments. In an aspect of the seventh embodiment, HetA is (i) a 5- or 6-membered heteroaromatic ring containing a total of from 1 to 4 heteroatoms independently selected from zero to 4 N, zero to 1 O and zero to 1 S, wherein each N is optionally in the form of an oxide, or (ii) a 9- or 10-membered bicyclic, fused ring system containing a total of from 1 to 4 heteroatoms independently selected from 1 to 4 N atoms, zero to 1 O atom, and zero to 1 S atom, wherein either one or both of the rings contain one or more of the heteroatoms, at least one ring is aromatic, each N is optionally in the form of an oxide, and S in a ring which is not aromatic is optionally S(O) or S(O)₂; wherein the heteroaromatic ring or the bicyclic, fused ring system is optionally substituted with a total of from 1 to 4 substituents as originally set forth for HetA in the seventh embodiment; HetB independently has the same definition as HetA; HetC independently has the same definition as HetA; and each HetD is independently a 5- or 6-membered heteroaromatic ring containing a total of from 1 to 4 heteroatoms independently selected from zero to 4 N atoms, zero to 10 atom, and zero to 1S, wherein each N is optionally in the form of an oxide, and wherein the heteroaromatic ring is optionally substituted with from 1 to 4 substituents each of which is independently Cl, Br, F, CN, NO₂, C₁₋₄ alkyl, C₁₋₄ fluoroalkyl, OH, O—C₁₋₄ alkyl, or O—C₁₋₄ fluoroalkyl.

An eighth embodiment of the present invention is a method as originally set forth above or as set forth in the first embodiment, wherein in the compound of Formula I, or a pharmaceutically acceptable salt thereof, each R^{A} is independently H or C₁₋₄ alkyl; and each R^{B} is independently H or C₁₋₄ alkyl; and all other variables are as originally defined or as defined in the second, third, fourth, fifth, sixth or seventh embodiments. In an aspect of this embodiment, each R^{A} is independently H or C₁₋₃ alkyl; and each R^{B} is independently H or C₁₋₃ alkyl. In another aspect, each R^{A} is independently H or CH₃; and each R^{B} is independently H or CH₃.

A ninth embodiment of the present invention is a method as originally set forth above or as set forth in the first embodiment, wherein in the compound of Formula I, or a pharmaceutically acceptable salt thereof, R¹ and R⁵ are as defined in the fourth embodiment; R² and R³ are as defined in the fifth embodiment; R⁴, R^{V} and R^{W} are as defined in the sixth embodiment; CycA, CycB, CycC, CycD, AryA, AryB, AryC, AryD, HetA, HetB, HetC, HetD, HetR, HetS and HetT are as defined in the seventh embodiment; and R^{A} and R^{B} are as defined in the eighth embodiment.

A tenth embodiment of the present invention is a method as set forth in the ninth embodiment, with the proviso that when R⁵ is H, then R¹ is:
(1) C(O)—C₁₋₄ alkyl,
(2) C(O)O—C₁₋₄ alkyl,
(3) C(O)N(R^{A})R^{B},
(4) S—C₁₋₄ alkyl,
(5) S(O)—C₁₋₄ alkyl,
(6) S(O)₂—C₁₋₄ alkyl,
(7) S(O)₂N(R^{A})R^{B},
(8) N(C₁₋₄ alkyl)S(O)₂—C₁₋₄ alkyl,
(9) N(C₁₋₄ alkyl)C(O)—C₁₋₄
(10) N(H)C(O)O—C₁₋₄ alkyl,
(11) N(C₁₋₄ alkyl)C(O)O—C₁₋₄ alkyl,
(12) N(H)S(O)₂N(R^{A})R^{B},
(13) N(C₁₋₄ alkyl)S(O)₂N(R^{A})R^{B},
(14) OC(O)N(R^{A})R^{B},
(15) N(H)C(O)N(R^{A})R^{B},
(16) N(C₁₋₄ alkyl)C(O)N(R^{A})R^{B},
(17) C₁₋₄ alkyl,
(18) C₁₋₄ fluoroalkyl,
(19) C₂₋₄ alkenyl,
(20) C₂₋₄ alkynyl,
(21) OH,
(22) O—C₁₋₄ alkyl,
(23) O—C₁₋₄ fluoroalkyl,
(24) C₁₋₄ alkyl substituted with OH, O—C₁₋₄ alkyl, O—C₁₋₄ fluoroalkyl, CN, NO₂, N(R^{A})R^{B}, C(O)N(R^{A})R^{B}, C(O)—C₁₋₄ alkyl, CO₂—C₁₋₄ alkyl, S—C₁₋₄ alkyl, S(O)—C₁₋₄ alkyl, S(O)₂—C₁₋₄ alkyl, S(O)₂N(R^{A})R^{B}, N(H)C(O)—C₁₋₄ alkyl, N(C₁₋₄ alkyl)C(O)—C₁₋₄ alkyl, N(H)CO₂—C₁₋₄ alkyl, N(C₁₋₄ alkyl)CO₂—C₁₋₄ alkyl, N(H)S(O)₂—C₁₋₄ alkyl, N(C₁₋₄ alkyl)S(O)₂—C₁₋₄ alkyl, N(H)S(O)₂N(R^{A})R^{B}, N(C₁₋₄ alkyl)S(O)₂N(R^{A})R^{B}, OC(O)N(R^{A})R^{B}, N(H)C(O)N(R^{A})R^{B}, or N(C₁₋₄ alkyl)C(O)N(R^{A})R^{B},
(25) CycA,
(26) AryA,
(27) HetA,
(28) HetR, or
(29) C₁₋₄ alkyl substituted with CycA, AryA, HetA, or HetR.

An eleventh embodiment of the present invention is a method as set forth in the ninth embodiment, with the proviso that when R⁵ is H, then R¹ is: (1) C(O)—C₁₋₄ alkyl, (2) C(O)O—C₁₋₄ alkyl, (3) C(O)N(R^{A})R^{B}, (4) S—C₁₋₄ alkyl, (5) S(O)—C₁₋₄ alkyl, (6) S(O)₂—C₁₋₄ alkyl, (7) S(O)₂N(R^{A})R^{B}, (8) N(H)C(O)O—C₁₋₄ alkyl, (9) N(C₁₋₄ alkyl)C(O)O—C₁₋₄ alkyl, (10) N(H)S(O)₂N(R^{A})R^{B}, (11) N(C₁₋₄ alkyl)S(O)₂N(R^{A})R^{B}, (12) OC(O)N(R^{A})R^{B}, (13) N(H)C(O)N(R^{A})R^{B}, (14) N(C₁₋₄ alkyl)C(O)N(R^{A})R^{B}, (15) C₁₋₄ alkyl, (16) C₁₋₄ fluoroalkyl, (17) C₂₋₄ alkenyl, (18) C₂₋₄ alkynyl, (19) OH, (20) O—C₁₋₄ alkyl, (21) O—C₁₋₄ fluoroalkyl,

(22) C₁₋₄ alkyl substituted with OH, O—C₁₋₄ alkyl, O—C₁₋₄ fluoroalkyl, CN, NO₂, N(R^{A})R^{B}, C(O)N(R^{A})R^{B}, C(O)—C₁₋₄ alkyl, CO₂—C₁₋₄ alkyl, S—C₁₋₄ alkyl, S(O)—C₁₋₄ alkyl, S(O)₂—C₁₋₄ alkyl, S(O)₂N(R^{A})R^{B}, N(H)C(O)—C₁₋₄ alkyl, N(C₁₋₄ alkyl)C(O)—C₁₋₄ alkyl, N(H)CO₂—C₁₋₄ alkyl, N(C₁₋₄ alkyl)CO₂—C₁₋₄ alkyl, N(H)S(O)₂—C₁₋₄ alkyl, N(C₁₋₄ alkyl)S(O)₂—C₁₋₄ alkyl, N(H)S(O)₂N(R^{A})R^{B}, N(C₁₋₄ alkyl)S(O)₂N(R^{A})R^{B}, OC(O)N(R^{A})R^{B}, N(H)C(O)N(R^{A})R^{B}, or N(C₁₋₄ alkyl)C(O)N(R^{A})R^{B}, (23) CycA, (24) AryA, (25) HetA, (26) HetR, or (27) C₁₋₄ alkyl substituted with CycA, AryA, HetA, or HetR.

A twelfth embodiment of the present invention is a method as originally set forth or as set forth in the first embodiment, wherein in the compound of Formula I, or a pharmaceutically acceptable salt thereof:

### R¹ is:

(1) Cl,
(2) Br,
(3) CN,
(4) C(O)CH₃,
(5) C(O)OCH₃,
(6) C(O)NH₂,
(9) S(O)₂CH₃,
(10) S(O)₂NH₂,
(11) NH₂,
(12) N(H)S(O)₂CH₃,
(13) N(H)C(O)CH₃,
(14) N(CH₃)S(O)₂CH₃,
(15) N(CH₃)C(O)CH₃,
(16) N(H)C(O)OCH₃,
(17) N(CH₃)C(O)OCH₃,
(18) N(H)S(O)₂NH₂,
(19) N(CH₃)S(O)₂NH₂,
(20) CH₃,
(21) CF₃,
(22) CH═CH₂,
(23) OCH₃,
(24) OCF₃,
(25) CycA,
(26) AryA,
(27) HetA,
(28) (CH₂)₁₋₃-CycA,
(29) (CH₂)₁₋₃-AryA, or
(30) (CH₂)₁₋₃-HetA;

### R² is:

(1) C₁₋₃ alkyl,
(2) (CH₂)₂₋₃OH,
(3) (CH₂)₂₋₃OCH₃,
(4) (CH₂)₂₋₃OCF₃,
(5) C₁₋₃ alkyl substituted with CN, NH₂, NH(CH₃), N(CH₃)₂, C(O)NH₂, C(O)NH(CH₃), C(O)N(CH₃)₂, C(O)CH₃, CO₂CH₃, SCH₃, S(O)CH₃, S(O)₂CH₃, S(O)₂NH₂, S(O)₂NH(CH₃), S(O)₂N(CH₃)₂, N(H)C(O)CH₃, or N(CH₃)C(O)CH₃,
(6) O—C₁₋₃ alkyl,
(7) C₃₋₆ cycloalkyl,
(8) HetS, or
(9) (CH₂)₁₋₃-HetS;

### R³ is H or CH₃;

alternatively R² and R³ together with the N atom to which they are attached form a saturated or mono-unsaturated heterocyclic ring selected from the group consisting of:

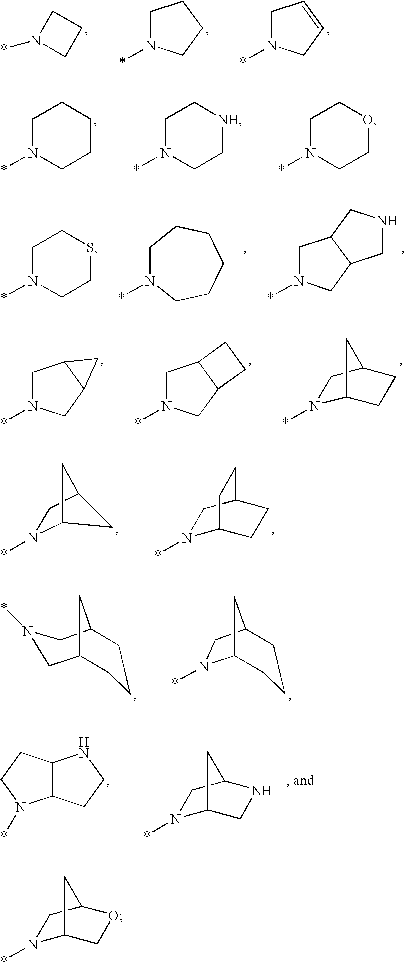

wherein the asterisk denotes the point of attachment of the heterocyclic ring to the rest of the molecule, and wherein the saturated or mono-unsaturated heterocyclic ring is optionally substituted with from 1 to 4 substituents each of which is independently Cl, Br, F, CN, CH₃, oxo, SO₂CH₃, OCH₃, CF₃, or CH₂OCH₃;

### R⁴ is:

(1) C(O)OC₁₋₃ alkyl,
(2) C(O)NH₂, or
(3) C(O)NR^{V}R^{W};

### R^{V} is H or CH₃;

### R^{W} is:

(1) C₁₋₃ alkyl,
(2) (CH₂)₂₋₃OH,
(3) (CH₂)₂₋₃OCH₃,
(3) (CH₂)₂₋₃OCF₃,
(4) C₁₋₃ alkyl substituted with CN, NH₂, NH(CH₃), N(CH₃)₂, C(O)NH₂, C(O)NH(CH₃), C(O)N(CH₃)₂, C(O)CH₃, CO₂CH₃, SCH₃, S(O)CH₃, S(O)₂CH₃, S(O)₂NH₂, S(O)₂NH(CH₃), S(O)₂N(CH₃)₂, N(H)C(O)CH₃, or N(CH₃)C(O)CH₃,
(5) CycC,
(6) AryC,
(7) HetC,
(8) HetT, or
(9) (CH₂)₁₋₃-CycC, (CH₂)₁₋₃-AryC, (CH₂)₁₋₃-HetC, or (CH₂)₁₋₃-HetT;

### R⁵ is H;

CycA is C₃₋₆ cycloalkyl;
AryA is phenyl which is optionally substituted with from 1 to 3 substituents each of which is independently Cl, Br, F, CH₃, OCH₃, CF₃, OCF₃, OCHF₂, OCH₂F, OH, SO₂CH₃, SO₂NH₂, C(O)NH(CH₃), or C(O)N(CH₃)₂;
HetA is a 5- or 6-membered heteroaromatic ring selected from the group consisting of pyrrolyl, thienyl, furanyl, pyrazolyl, imidazolyl, triazolyl, tetrazolyl, oxazolyl, thiazolyl, isoxazolyl, isothiazolyl, oxadiazolyl, pyridinyl, pyrazinyl, and pyrimidinyl, wherein the heteroaromatic ring is optionally substituted with a total of from 1 to 3 substituents, each of which is independently Cl, Br, F, CH₃, or OCH₃;
CycC independently has the same definition as CycA;
AryC independently has the same definition as AryA;
HetC is (i) a 5- or 6-membered heteroaromatic ring selected from the group consisting of pyrrolyl, thienyl, furanyl, pyrazolyl, imidazolyl, triazolyl, tetrazolyl, oxazolyl, thiazolyl, isoxazolyl, isothiazolyl, oxadiazolyl, pyridinyl, pyrazinyl, and pyrimidinyl or (ii) a bicyclic, fused ring system selected from the group consisting of 2,3-dihydrobenzo-1,4-dioxinyl, benzo-1,3-dioxolyl, quinolinyl, isoquinolinyl, quinazolinyl, naphthyridinyl, benzoxazinyl, cinnolinyl, and 4H-imidazo[4,5-b]pyridinyl; wherein the heteroaromatic ring or the bicyclic, fused ring system is optionally substituted with a total of from 1 to 3 substituents, wherein from zero to 3 substituents are each independently Cl, Br, F, CH₃, or OCH₃, and from zero to 1 substituent is phenyl;
HetS is a saturated heterocyclic ring selected from the group consisting of pyrrolidinyl, tetrahydrofuranyl, piperidinyl, piperazinyl, morpholinyl, and thiomorpholinyl, wherein the saturated heterocyclic ring is optionally substituted with from 1 to 4 substituents each of which is independently Cl, Br, F, CH₃, oxo, OCH₃, CF₃, SO₂CH₃, or CH₂OCH₃; and
HetT independently has the same definition as HetS.

A thirteenth embodiment of the present invention is identical to the twelfth embodiment, except that R² and R³ together with the N atom to which they are attached form a heterocyclic ring selected from the group consisting of:

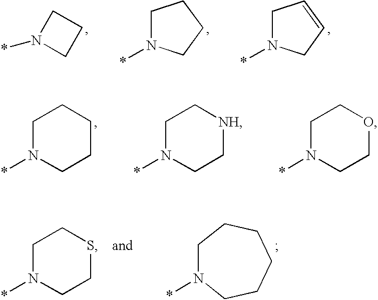

wherein the asterisk denotes the point of attachment of the heterocyclic ring to the rest of the molecule, and wherein the heterocyclic ring is optionally substituted with from 1 to 4 substituents each of which is independently Cl, Br, F, CN, CH₃, oxo, C(O)CH₃, CO₂CH₃, SO₂CH₃, OCH₃, CF₃, or CH₂OCH₃;

A fourteenth embodiment of the present invention is a method as originally set forth or as set forth in the first embodiment, wherein in the compound of Formula I, or a pharmaceutically acceptable salt thereof:

### R¹ is Cl or Br;

### R² is:

(1) C₁₋₃ alkyl,
(2) (CH₂)₂₋₃OH,
(3) (CH₂)₂₋₃OCH₃,
(4) (CH₂)₁₋₂NH₂, (CH₂)₁₋₂C(O)NH₂, or (CH₂)₁₋₂S(O)₂NH₂,
(5) OCH₃,
(6) C₃₋₆ cycloalkyl, or
(7) CH₂-HetS;

### R³ is H or CH₃;

alternatively R² and R³ together with the N atom to which they are attached form a saturated or mono-unsaturated heterocyclic ring selected from the group consisting of:

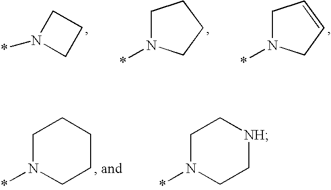

wherein the saturated or mono-unsaturated heterocyclic ring is optionally substituted with from 1 to 4 substituents each of which is independently Cl, Br, F, CN, CH₃, oxo, OCH₃, CF₃, or CH₂OCH₃;
R⁴ is C(O)OCH₃, C(O)OCH₂CH₃, C(O)NH₂, C(O)N(H)CH₂CH₂OH, C(O)N(H)CH₂CH₂OCH₃, C(O)N(H)(CH₂)₁₋₃-AryC, C(O)N(H)(CH₂)₁₋₃-HetC, or C(O)N(H)(CH₂)₁₋₃-HetT;
HetS is a saturated heterocyclic ring selected from the group consisting of pyrrolidinyl, tetrahydrofuranyl, piperidinyl, piperazinyl, morpholinyl, and thiomorpholinyl, wherein the saturated heterocyclic ring is optionally substituted with from 1 to 4 substituents each of which is independently Cl, Br, F, CH₃, oxo, OCH₃, CF₃, SO₂CH₃, or CH₂OCH₃; and
HetT independently has the same definition as HetS.

A fourteenth embodiment of the present invention is a method as originally set forth or as set forth in the first embodiment, wherein the compound of Formula I, or a pharmaceutically acceptable salt thereof, administered to the subject is selected from the group consisting of the compounds set forth in Examples 1 to 68 below.

A fifteenth embodiment of the present invention is a method as originally set forth or as set forth in the first embodiment, wherein the compound of Formula I, or a pharmaceutically acceptable salt thereof, administered to the subject is selected from the group consisting of the compounds set forth in Examples 1 and 13 to 68 below.

In the method of the invention as originally described in the Summary of the Invention or as described in any of the foregoing embodiments, or aspects thereof, the compound of Formula I, or a pharmaceutically acceptable salt thereof, can be administered per se or as an active ingredient of a pharmaceutical composition comprising a pharmaceutically acceptable carrier.

Still other embodiments of the present invention include the following:

(a) A method for inhibition of HIV reverse transcriptase, for treatment or prophylaxis of HIV infection, or for treatment, prophylaxis or delay in the onset of AIDS, which comprises administering to a subject in need thereof a compound of Formula I, or a pharmaceutically acceptable salt thereof, in combination with another anti-HIV agent selected from the group consisting of HIV antiviral agents, immunomodulators, and anti-infective agents; wherein the compound of Formula I and the anti-HIV agent are each employed in an amount that renders the combination effective for inhibition of HIV reverse transcriptase, for treatment or prophylaxis of infection by HIV, or for treatment, prophylaxis or delay in the onset of AIDS.

(b) The method of (a), wherein the other anti-HIV agent is selected from the group consisting of HIV protease inhibitors, HIV reverse transcriptase inhibitors other than a compound of Formula I, and HIV integrase inhibitors.

(c) A method for inhibition of HIV reverse transcriptase, for treatment or prophylaxis of HIV infection, or for treatment, prophylaxis or delay in the onset of AIDS, which comprises administering to a subject in need thereof a pharmaceutical composition comprising an effective amount of a compound of Formula I, or a pharmaceutically acceptable salt thereof, and a pharmaceutically acceptable carrier.

(d) A method for inhibition of HIV reverse transcriptase, for treatment or prophylaxis of HIV infection, or for treatment, prophylaxis or delay in the onset of AIDS, which comprises administering to a subject in need thereof a combination of (i) a pharmaceutical composition comprising a compound of Formula I, or a pharmaceutically acceptable salt thereof, and a pharmaceutically acceptable carrier and (ii) an another anti-HIV agent selected from the group consisting of HIV antiviral agents, immunomodulators, and anti-infective agents; wherein the compound of Formula I and the other anti-HIV agent are each employed in an amount that renders the combination effective for inhibition of HIV reverse transcriptase, for treatment or prophylaxis of infection by HIV, or for treatment, prophylaxis or delay in the onset of AIDS.

Additional embodiments of the invention include the methods set forth in embodiments (a)-(d) above, wherein the compound of Formula I employed therein is a compound as defined above in one of the earlier-described embodiments (or aspects thereof) of the method of the present invention.

In the methods of the present invention involving a combination of active compounds (e.g., a compound of Formula I and another HIV antiviral agent), it is understood that the active compounds can be administered separately or together, and when administered separately, the active compounds can be given concurrently or at different times (e.g., alternately). When the active compounds are administered together (either per se or more typically in a pharmaceutical composition), they can both be part of a single composition (e.g., an admixture of the compounds optionally including one or more excipients) or they can be in separate compositions (e.g., encapsulated compositions respectively containing one of the active compounds and optionally one or more excipients) that can be packaged together or separately.

The present invention also includes a compound of Formula I (i) for use in, (ii) for use as a medicament for, or (iii) for use in the preparation of a medicament for: (a) inhibition of HIV reverse transcriptase, (b) treatment or prophylaxis of infection by HIV, or (c) treatment, prophylaxis or delay in the onset of AIDS. In these uses, the compounds of the present invention can optionally be employed in combination with one or more other anti-HIV agents selected from HIV antiviral agents, anti-infective agents, and immunomodulators.

Additional embodiments of the invention include the uses set forth in the preceding paragraph, wherein the compound of Formula I employed therein is a compound as defined in one of the earlier-described embodiments (or aspects thereof) of the method of the present invention. In all of these embodiments, the compound can optionally be used in the form of a pharmaceutically acceptable salt and can be employed per se or as an active ingredient in a pharmaceutical composition comprising a pharmaceutically acceptable carrier.

Additional embodiments of the invention include each of the methods and uses as originally described and as set forth in the earlier-described embodiments (or aspects thereof), wherein the compound of Formula I or its pharmaceutically acceptable salt employed therein is substantially pure. As used herein “substantially pure” means that the compound or its salt is present (e.g., in a product isolated from a chemical reaction or a metabolic process) in an amount of at least about 90 wt. % (e.g., from about 95 wt. % to 100 wt. %), preferably at least about 95 wt. % (e.g., from about 98 wt. % to 100 wt. %), more preferably at least about 99 wt. %, and most preferably 100 wt. %. The level of purity of the compounds and salts can be determined using standard methods of analysis. A compound or salt of 100% purity can alternatively be described as one which is free of detectable impurities as determined by one or more standard methods of analysis. With respect to a compound of the invention which has one or more asymmetric centers and can occur as mixtures of stereoisomers, a substantially pure compound can be either a substantially pure mixture of the stereoisomers or a substantially pure individual diastereomer or enantiomer. With respect to a pharmaceutical composition comprising a compound of Formula I or its salt and a pharmaceutically acceptable carrier and optionally one or more excipients, the term “substantially pure” is in reference to Compound I or its salt per se; i.e., the purity of the active ingredient in the composition.

The present invention also includes a compound, or a pharmaceutically acceptable salt thereof, selected from the group consisting of the compounds set forth in Examples 1 and 13 to 68 below. An embodiment of the invention is a compound, or a pharmaceutically acceptable salt thereof, selected from the group consisting of the compounds set forth in Examples 1 and 13 to 68 below, wherein the compound is substantially pure.

As used herein, the term “alkyl” refers to any linear or branched chain alkyl group having a number of carbon atoms in the specified range. Thus, for example, “C₁₋₆ alkyl” (or “C₁-C₆ alkyl”) refers to any of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl.

The term “alkylene” refers to any divalent linear or branched chain aliphatic hydrocarbon radical (or alternatively an “alkanediyl”) having a number of carbon atoms in the specified range. Thus, for example, “—C₁₋₆ alkylene-” refers to any of the C₁ to C₆ linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—. Another sub-class of interest an alkylene selected from the group consisting of —CH₂—, —CH(CH₃)—, and —C(CH₃)₂—.

The term “cycloalkyl” refers to any cyclic ring of an alkane having a number of carbon atoms in the specified range. Thus, for example, “C₃₋₈ cycloalkyl” (or “C₃-C₈ cycloalkyl”) refers to cyclopropyl, cyclobutyl, cyclopentyl, cyclohexyl, cycloheptyl, and cyclooctyl.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms has been replaced with a halogen (i.e., F, Cl, Br and/or I). Thus, for example, “C₁₋₆ haloalkyl” (or “C₁-C₆ haloalkyl”) refers to a C₁ to C₆ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.). A fluoroalkyl of particular interest is CF₃.

The term “C(O)” appearing in the definition of a functional group (e.g., “C(O)R^{A}”) refers to carbonyl. The term “S(O)₂” or “SO₂” appearing in the definition of a functional group refers to sulfonyl, the term “S(O)” refers to sulfinyl, and the terms “C(O)O” and “CO₂” both refer to carboxyl.

The left-most atom or variable shown in any of the groups in the definitions of R¹ to R⁵ is the atom or variable attached to or nearest to the indole ring. Thus, for example, a compound of the present invention in which R¹ is J-AryA, J is C(O)N(R^{A}), R⁴ is C(O)NR^{V}R^{W}, R⁵ is H, R² is O—C₁₋₆ alkyl, and R³ is C₁₋₆ alkyl is as follows:

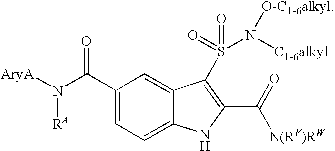

The symbols “*” and “ [symbol] ” at the end of a bond each refer to the point of attachment of a functional group or other chemical moiety to the rest of the molecule of which it is a part.

Unless expressly stated to the contrary in a particular context, any of the various carbocyclic and heterocyclic rings and ring systems defined herein may be attached to the rest of the compound at any ring atom (i.e., any carbon atom or any heteroatom) provided that a stable compound results. Suitable aryls include phenyl, 9- and 10-membered bicyclic, fused carbocyclic ring systems, and 11- to 14-membered tricyclic fused carbocyclic ring systems, wherein in the fused carbocyclic ring systems at least one ring is aromatic. Suitable aryls include, for example, phenyl, naphthyl, tetrahydronaphthyl (tetralinyl), indenyl, anthracenyl, and fluorenyl. Suitable heteroaryls include 5- and 6-membered heteroaromatic rings and 9- and 10-membered bicyclic, fused ring systems in which at least one ring is aromatic, wherein the heteroaromatic ring or the bicyclic, fused ring system contains from 1 to 4 heteroatoms independently selected from N, O and S, wherein each N is optionally in the form of an oxide and each S in a ring which is not aromatic is optionally S(O) or S(O)₂. Suitable 5- and 6-membered heteroaromatic rings include, for example, pyridyl, pyrrolyl, pyrazinyl, pyrimidinyl, pyridazinyl, triazinyl, thienyl, furanyl, imidazolyl, pyrazolyl, triazolyl, tetrazolyl, oxazolyl, isooxazolyl, oxadiazolyl, oxatriazolyl, thiazolyl, isothiazolyl, and thiadiazolyl. Suitable heterobicyclic, fused ring systems include, for example, benzofuranyl, indolyl, indazolyl, naphthyridinyl, isobenzofuranyl, benzopiperidinyl, benzisoxazolyl, benzoxazolyl, chromenyl, quinolinyl, isoquinolinyl, cinnolinyl, quinazolinyl, tetrahydroquinolinyl, tetrahydroisoquinolinyl, isoindolyl, benzodioxolyl (e.g., benzo-1,3-dioxolyl:

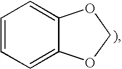

benzopiperidinyl, benzisoxazolyl, benzoxazolyl, benzoxazinyl, chromanyl, isochromanyl, benzothienyl, benzofuranyl, imidazo[1,2-a]pyridinyl, benzotriazolyl, dihydroindolyl, dihydroisoindolyl, indazolyl, indolinyl, isoindolinyl, quinoxalinyl, quinazolinyl, 2,3-dihydrobenzofuranyl, 4H-imidazo[4,5-b]pyridinyl (i.e.,

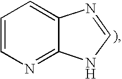

and 2,3-dihydrobenzo-1,4-dioxinyl (i.e.,

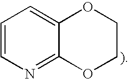

Suitable saturated and mono-unsaturated heterocyclic rings include 4- to 7-membered saturated and mono-unsaturated heterocyclic rings containing at least one carbon atom and from 1 to 4 heteroatoms independently selected from N, O and S, wherein each S is optionally oxidized to S(O) or S(O)₂. Suitable 4- to 7-membered saturated heterocyclics include, for example, azetidinyl, piperidinyl, morpholinyl, thiomorpholinyl, thiazolidinyl, isothiazolidinyl, oxazolidinyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, and dioxanyl. Suitable mono-unsaturated heterocyclic rings include those corresponding to the saturated heterocyclic rings listed in the preceding sentence in which a single bond is replaced with a double bond (e.g., a carbon-carbon single bond is replaced with a carbon-carbon double bond). Suitable saturated and mono-unsaturated heterobicyclic rings include 6- to 10-membered saturated and mono-unsaturated, bridged or fused heterobicyclic rings containing from 1 to 4 heteroatoms independently selected from N, O and S, where each S is optionally oxidized to S(O) or S(O)₂. Suitable saturated heterobicyclics include those disclosed elsewhere (see, e.g., the definition of R²+R³ in the twelfth embodiment of the invention), and suitable mono-unsaturated heterobicyclics include those corresponding to the saturated heterobicyclics disclosed elsewhere in which a single bond is replaced with a double bond. It is understood that the specific rings and ring systems suitable for use in the present invention are not limited to those listed in this paragraph. The rings and ring systems listed in this paragraph are merely representative.

Unless expressly stated to the contrary, all ranges cited herein are inclusive. For example, a heterocyclic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, and 4 heteroatoms. As another example, an aryl or heteroaryl described as optionally substituted with “from 1 to 5 substituents” is intended to include as aspects thereof, an aryl or heteroaryl optionally substituted with 1 to 4 substituents, 1 to 3 substituents, 1 to 2 substituents, 2 to 5 substituents, 2 to 4 substituents, 2 to 3 substituents, 3 to 5 substituents, 3 to 4 substituents, 4 to 5 substituents, 1 substituent, 2 substituents, 3 substituents, 4 substituents, and 5 substituents.

When any variable (e.g., R^{A}, R^{B}, AryD, or HetD) occurs more than one time in any constituent or in Formula I or in any other formula depicting and describing compounds employed in the invention, its definition on each occurrence is independent of its definition at every other occurrence. Also, combinations of substituents and/or variables are permissible only if such combinations result in stable compounds.

The term “substituted” (e.g., as in “is optionally substituted with from 1 to 5 substituents.”) includes mono- and poly-substitution by a named substituent to the extent such single and multiple substitution (including multiple substitution at the same site) is chemically allowed. Unless expressly stated to the contrary, substitution by a named substituent is permitted on any atom in a ring (e.g., cycloalkyl, aryl, or heteroaryl) provided such ring substitution is chemically allowed and results in a stable compound. Ring substituents can be attached to the ring atom which is attached to the rest of the molecule.

As a result of the selection of substituents and substituent patterns, certain compounds of the present invention can exhibit keto-enol tautomerism. All tautomeric forms of these compounds, whether individually or in mixtures, are within the scope of the present invention. For example, in instances where a hydroxy (—OH) substituent(s) is (are) permitted on a heteroaromatic ring and keto-enol tautomerism is possible, it is understood that the substituent might in fact be present, in whole or in part, in the keto form, as exemplified here for a hydroxypyridinyl substituent:

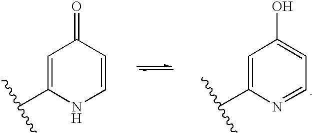

Compounds of the present invention having a hydroxy substituent on a carbon atom of a heteroaromatic ring are understood to include compounds in which only the hydroxy is present, compounds in which only the tautomeric keto form (i.e., an oxo substitutent) is present, and compounds in which the keto and enol forms are both present.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

As a result of the selection of substituents and substituent patterns, certain compounds employed in the present invention can have asymmetric centers and can occur as mixtures of stereoisomers, or as individual diastereomers, or enantiomers. All isomeric forms of these compounds, whether individually or in mixtures, are within the scope of the present invention.

The present invention involves the use of (i) compounds embraced by Formula I in the inhibition of HIV reverse transcriptase (wild type and/or mutant strains thereof), the prophylaxis or treatment of infection by human immunodeficiency virus (HIV) and the prophylaxis, treatment or delay in the onset of consequent pathological conditions such as AIDS. Prophylaxis of AIDS, treating AIDS, delaying the onset of AIDS, or treating or prophylaxis of infection by HIV is defined as including, but not limited to, treatment of a wide range of states of HIV infection: AIDS, ARC (AIDS related complex), both symptomatic and asymptomatic, and actual or potential exposure to HIV. For example, the present invention can be employed to treat infection by HIV after suspected past exposure to HIV by such means as blood transfusion, exchange of body fluids, bites, accidental needle stick, or exposure to patient blood during surgery. As another example, the present invention can also be employed to prevent transmission of HIV from a pregnant female infected with HIV to her unborn child or from an REV-infected female who is nursing (i.e., breast feeding) a child to the child via administration of an effective amount of a compound of Formula I, or a pharmaceutically acceptable salt thereof.

The compounds can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. Certain of the compounds employed in the present invention carry an acidic moiety (e.g., —COOH or a phenolic group), in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The term “administration” and variants thereof (e.g., “administering” a compound) in reference to a compound of Formula I mean providing the compound or a prodrug of the compound to the individual in need of treatment or prophylaxis. When a compound or a prodrug thereof is provided in combination with one or more other active agents (e.g., antiviral agents useful for treating or prophylaxis of HIV infection or AIDS), “administration” and its variants are each understood to include provision of the compound or prodrug and other agents at the same time or at different times. When the agents of a combination are administered at the same time, they can be administered together in a single composition or they can be administered separately.

As used herein, the term “composition” is intended to encompass a product comprising the specified ingredients, as well as any product which results, directly or indirectly, from combining the specified ingredients.

By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV reverse transcriptase (wild type and/or mutant strains thereof) and thereby elicit the response being sought (i.e., an “inhibition effective amount”). When the active compound (i.e., active ingredient) is administered as the salt, references to the amount of active ingredient are to the free form (i.e., the non-salt form) of the compound.

In the method of the present invention (i.e., inhibiting HIV reverse transcriptase, treating or prophylaxis of HIV infection or treating, prophylaxis of, or delaying the onset of AIDS), the compounds of Formula I, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds employed in the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990.

The compounds of Formula I can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As noted above, the present invention is also directed to the use of the compounds of Formula I in combination with one or more agents useful in the treatment of HIV infection or AIDS. For example, the compounds of Formula I can be effectively administered, whether at periods of pre-exposure and/or post-exposure, in combination with effective amounts of one or more HIV antiviral agents, immunomodulators, antiinfectives, or vaccines useful for treating HIV infection or AIDS, such as those disclosed in Table 1 of WO 01/38332 or in the Table in WO 02/30930. Suitable HIV antiviral agents for use in combination with the compounds of Formula I include, for example, HIV protease inhibitors (e.g., indinavir, atazanavir, lopinavir optionally with ritonavir, saquinavir, or nelfinavir), nucleoside HIV reverse transcriptase inhibitors (e.g., abacavir, lamivudine (3TC), zidovudine (AZT), or tenofovir), non-nucleoside HIV reverse transcriptase inhibitors (e.g., efavirenz or nevirapine), and HIV integrase inhibitors such as those described in WO 02/30930, WO 03/35076, and WO 03/35077. It will be understood that the scope of combinations of compounds of Formula I with HIV antiviral agents, immunomodulators, anti-infectives or vaccines is not limited to the foregoing substances or to the list in the above-referenced Tables in WO 01/38332 and WO 02/30930, but includes in principle any combination with any pharmaceutical composition useful for the treatment of HIV infection or AIDS. The HIV antiviral agents and other agents will typically be employed in these combinations in their conventional dosage ranges and regimens as reported in the art, including, for example, the dosages described in the Physicians' Desk Reference, 58^{th} edition, Thomson PDR, 2004. The dosage ranges for a compound of Formula I in these combinations are the same as those set forth above. It is understood that pharmaceutically acceptable salts of the compounds employed in the invention and/or the other agents (e.g., indinavir sulfate) can be used as well.

Abbreviations employed herein include the following:
Ac=acetyl
CHAPS=3[(3-cholamidopropyl)dimethylammonio]-propanesulfonic acid
dGTP=deoxyguanosine triphosphate
DCM=dichloromethane
DIEA=diisopropylethylamine
DMF=N,N-dimethylformamide
DMSO=dimethyl sulfoxide
dNTP=deoxynucleoside triphosphate
dppf=1,1′-bis(diphenylphosphino)ferrocene
EDTA=ethylenediaminetetracetic acid
EGTA=ethylene glycol bis(2-aminoethyl ether)-N,N,N′,N′-tetraacetic acid
ES MS=electrospray mass spectroscopy
Et=ethyl
HOBT or HOBt=1-hydroxy benzotriazole hydrate
LCMS=liquid chromatography mass spectroscopy
Me=methyl
MeOH=methanol
5-Naph=naphth-5-yl
NMR=nuclear magnetic resonance
Ph=phenyl
PS-DIEA=polystyrene diisopropylethylamine
PS-DMAP=polystyrene 4-N,N-dimethylaminopyridine
PS-DCC=polystyrene dicyclohexylcarbodiimide
Ph=phenyl
TFA=trifluoroacteic acid
THF=tetrahydrofuran

The compounds employed in the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds employed in the invention will be readily apparent to the person of ordinary skill in the art in light of the following reaction schemes and examples. Unless otherwise indicated, all variables are as defined above.

Scheme 1 depicts a general method for preparing indole-2-carboxamide compounds employed in the present invention where carboxylic ester 1 is treated with a suitable primary or secondary amine in the presence of a tertiary amine base (e.g., DIEA) in a suitable organic solvent. This treatment generates the sulfonamide with concomitant cleavage of the indole 1-phenylsulphonyl protecting group to furnish 2. Direct treatment of 2 with ammonia at elevated temperature provides primary amide 3, and, alternatively, hydrolysis of 2 affords the carboxylic acid 4 which can be coupled with an amine (using, e.g., PS-DCC, HOBt and DIEA) to provide amide 5. Compound 3 can be converted to 6 using palladium catalysis under standard Suzuki conditions (e.g., Cl₂Pd(dppf)₂, CsCO₃, THF/H₂O), and can be converted to 7 using modified cyanation conditions (for 6, Pd(OAc)₂, PS—PPh₃, Zn(CN)₂).

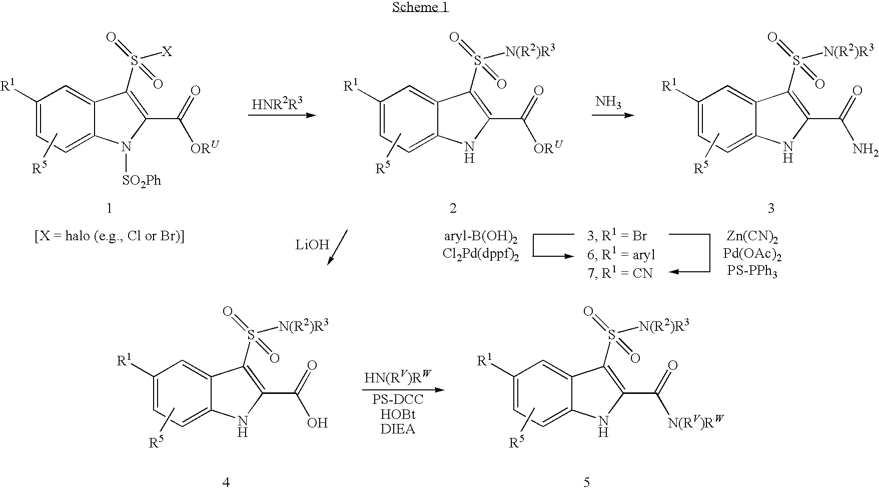

Indole-2-carboxylates of formula I can be prepared using procedures described in WO 2004/014851. Amines suitable for use in accordance with Scheme 1 can either be obtained from commercial sources or can be prepared using the methods known in the art, such as those described in Richard Larock, Comprehensive Organic Transformations, VCH Publishers Inc, 1989, pp 385-438.

In the processes for preparing compounds set forth in the foregoing scheme, functional groups in various moieties and substituents may be sensitive or reactive under the reaction conditions employed and/or in the presence of the reagents employed. Such sensitivity/reactivity can interfere with the progress of the desired reaction to reduce the yield of the desired product, or possibly even preclude its formation. Accordingly, it may be necessary or desirable to protect sensitive or reactive groups on any of the molecules concerned. Protection can be achieved by means of conventional protecting groups, such as those described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 3^{rd} edition, 1999, and 2^{nd} edition, 1991. The protecting groups may be removed at a convenient subsequent stage using methods known in the art. Alternatively the interfering group can be introduced into the molecule subsequent to the reaction step of concern.

The following examples serve only to illustrate the invention and its practice. The examples are not to be construed as limitations on the scope or spirit of the invention.

### Example 1

### 5-Bromo-3-(pyrrolidin-1-ylsulfonyl)-1H-indole-2-carboxamide

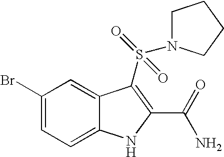

A mixture of ethyl 5-bromo-3-(chlorosulfonyl)-1-(phenylsulfonyl)-1H-indole-2-carboxylate (Dinsmore, C. J., et al., PCT Int. Appl. (2004) WO 2004014300) (51 mg, 0.1 mmol), pyrrolidine (13 μL, 0.15 mmol), and DIEA (49 μL, 0.3 mmol) in DCM (2 mL) was shaken 1 hour at room temperature. After this time, the solution was concentrated under a stream of nitrogen and the residue was re-dissolved in 2M NH₃-MeOH. The resulting mixture was heated at 100° C. overnight before being cooled to room temperature and concentrated under reduced pressure. The concentrated residue was purified by LCMS to give the desired product as a slightly yellow solid. Analytical LCMS: single peak (214 nm), 3.012 min, ES MS (M+1)=372; ¹HNMR (500 MHz, d₆-DMSO) δ 12.59 (br, 1H), 8.38 (s, 1H), 8.22 (s, 1H), 8.12 (d, J=1.9 Hz, 1H), 7.51 (d, J=8.7 Hz, 1H), 7.47 (dd, J=8.7, 1.9 Hz, 1H), 3.21-3.11 (m, 4H), 1.71-1.61 (m, 4H); FIRMS, calc'd for C₁₃H₁₄BrN₃O₃S (M+1), 372.0012. found 372.0015.

### Examples 2-46

The compounds in the following table were prepared in accordance with the procedures set forth in Example 1, or routine variations thereof, using the appropriate amine in place of pyrrolidine and, if necessary, the appropriate amine in place of ammonia.

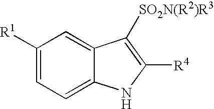

| | | | | | ES MS |
|---|---|---|---|---|---|
| Ex. | Name | R¹ | N(R²)R² | R⁴ | (M + 1) |
| 2 | 5-chloro-3- [(cyclohexylamino)sulfonyl]-1H- indole-2-carboxamide | Cl | 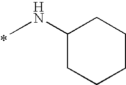 | C(O)NH₂ | 356.8 |
| 3 | 5-chloro-3- [(cyclobutylamino)sulfonyl]-1H- indole-2-carboxamide | Cl | 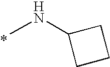 | C(O)NH₂ | 328.8 |
| 4 | 5-chloro-3- [(cyclopentylamino)sulfonyl]-1H- indole-2-carboxamide | Cl | 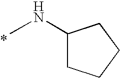 | C(O)NH₂ | 342.8 |
| 5 | 5-chloro-3-(piperidin-1-ylsulfonyl)-1H- indole-2-carboxamide | Cl | 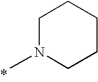 | C(O)NH₂ | 342.8 |
| 6 | 5-chloro-3-(pyrrolidin-1-ylsulfonyl)- 1H-indole-2-carboxamide | Cl | 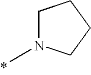 | C(O)NH₂ | 328.8 |
| 7 | 3-(azetidin-1-ylsulfonyl)-5-chloro-1H- indole-2-carboxamide | Cl | 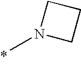 | C(O)NH₂ | 314.8 |
| 8 | 5-bromo-3- {[cyclopropyl(methyl)amino]sulfonyl}- 1H-indole-2-carboxamide | Br | 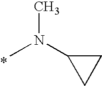 | C(O)NH₂ | 373.2 |
| 9 | 3-({[2- (aminosulfonyl)ethyl]amino}sulfonyl)- 5-bromo-1H-indole-2-carboxamide | Br | N(H)(CH₂)₂SO₂NH₂ | C(O)NH₂ | 426.3 |
| 10 | 5-bromo-3-(2,5-dihydro-1H-pyrrol-1- ylsulfonyl)-1H-indole-2-carboxamide | Br | 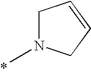 | C(O)NH₂ | 371.2 |
| 11 | 5-bromo-3- [(cyclopropylamino)sulfonyl]-1H- indole-2-carboxamide | Br | 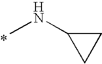 | C(O)NH₂ | 359.2 |
| 12 | 5-bromo-3- {[methoxy(methyl)amino]sulfonyl}- 1H-indole-2-carboxamide | Br | N(Me)OMe | C(O)NH₂ | 363.2 |
| 13 | 5-bromo-3-(piperidin-1-ylsulfonyl)-1H- indole-2-carboxamide | Br | 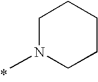 | C(O)NH₂ | 387.3 |
| 14 | 5-bromo-3-(pyrrolidin-1-ylsulfonyl)- 1H-indole-2-carboxamide | Br | 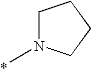 | C(O)NH₂ | 373.2 |
| 15 | 5-bromo-3-{[(2S)-2- (methoxymethyl)pyrrolidin-1- yl]sulfonyl}-1H-indole-2-carboxamide | Br | 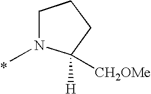 | C(O)NH₂ | 417.3 |
| 16 | 5-bromo-3- [(cyclohexylamino)sulfonyl]-1H- indole-2-carboxamide | Br | 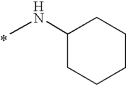 | C(O)NH₂ | 401.3 |
| 17 | 5-bromo-3- [(cyclopentylamino)sulfonyl]-1H- indole-2-carboxamide | Br | 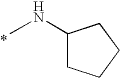 | C(O)NH₂ | 387.3 |
| 18 | 5-bromo-3-{[(tetrahydrofuran-2- ylmethyl)amino]sulfonyl}-1H-indole-2- carboxamide | Br | 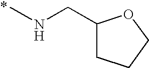 | C(O)NH₂ | 403.3 |
| 19 | 5-bromo-3-{[(2S)-2- (trifluoromethyl)pyrrolidin-1- yl]sulfonyl}-1H-indole-2-carboxamide | Br | 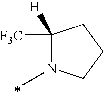 | C(O)NH₂ | 441.2 |
| 20 | 5-bromo-3-[(3-methoxypiperidin-1- yl)sulfonyl]-1H-indole-2-carboxamide | Br | 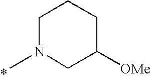 | C(O)NH₂ | 417.3 |
| 21 | 5-bromo-3-[(3,3-difluoropiperidin-1- yl)sulfonyl]-1H-indole-2-carboxamide | Br | 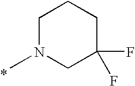 | C(O)NH₂ | 423.3 |
| 22 | 5-bromo-3-[(3-fluoropyrrolidin-1- yl)sulfonyl]-1H-indole-2-carboxamide | Br | 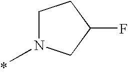 | C(O)NH₂ | 391.2 |
| 23 | 5-bromo-3-[(propylamino)sulfonyl]- 1H-indole-2-carboxamide | Br | N(H)CH₂CH₂Me | C(O)NH₂ | 361.2 |
| 24 | methyl 5-bromo-3-(piperidin-1- ylsulfonyl)-1H-indole-2-carboxylate | Br | 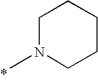 | C(O)OMe | 402.3 |
| 25 | methyl 5-bromo-3-(pyrrolidin-1- ylsulfonyl)-1H-indole-2-carboxylate | Br | 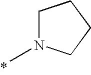 | C(O)OMe | 388.3 |
| 26 | methyl 5-bromo-3- [(cyclopentylamino)sulfonyl]-1H- indole-2-carboxylate | Br | 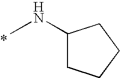 | C(O)OMe | 402.3 |
| 27 | ethyl 5-bromo-3-(piperidin-1- ylsulfonyl)-1H-indole-2-carboxylate | Br | 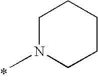 | C(O)OEt | 416.3 |
| 28 | ethyl 5-bromo-3-{[(2- methoxyethyl)amino]sulfonyl}-1H- indole-2-carboxylate | Br | N(H)CH₂CH₂OMe | C(O)OEt | 406.3 |
| 29 | ethyl 5-bromo-3- [(cyclopentylamino)sulfonyl]-1H- indole-2-carboxylate | Br | 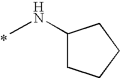 | C(O)OEt | 416.3 |
| 30 | ethyl 5-bromo-3-{[2- (trifluoromethyl)pyrrolidin-1- yl]sulfonyl}-1H-indole-2-carboxylate | Br | 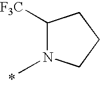 | C(O)OEt | 470.3 |
| 31 | 5-bromo-N-(2-hydroxyethyl)-3- (piperidin-1-ylsulfonyl)-1H-indole-2- carboxamide | Br | 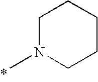 | C(O)NHCH₂CH₂OH | 431.3 |
| 32 | 5-bromo-N-(2-hydroxyethyl)-3- (pyrrolidin-1-ylsulfonyl)-1H-indole-2- carboxamide | Br | 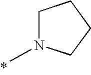 | C(O)NHCH₂CH₂OH | 417.3 |
| 33 | 5-bromo-3- [(cyclopropylamino)sulfonyl]-N-(2- hydroxyethyl)-1H-indole-2- carboxamide | Br | 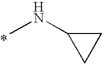 | C(O)NHCH₂CH₂OH | 403.3 |
| 34 | 5-bromo-3- [(cyclohexylamino)sulfonyl]-N-(2- hydroxyethyl)-1H-indole-2- carboxamide | Br | 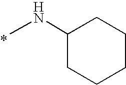 | C(O)NHCH₂CH₂OH | 445.4 |
| 35 | 5-bromo-3- [(cyclopentylamino)sulfonyl]-N-(2- hydroxyethyl)-1H-indole-2- carboxamide | Br | 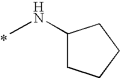 | C(O)NHCH₂CH₂OH | 431.3 |
| 36 | 5-bromo-N-(2-hydroxyethyl)-3-[(3- oxopiperazin-1-yl)sulfonyl]-1H-indole- 2-carboxamide | Br | 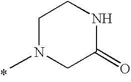 | C(O)NHCH₂CH₂OH | 446.3 |
| 37 | 5-chloro-3-(2,5-dihydro-1H-pyrrol-1- ylsulfonyl)-1H-indole-2-carboxamide | Cl | 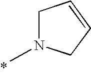 | C(O)NH₂ | 326.8 |
| 38 | 5-chloro-3-[(3-fluoropyrrolidin-1- yl)sulfonyl]-1H-indole-2-carboxamide | Cl | 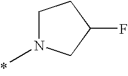 | C(O)NH₂ | 346.8 |
| 39 | 5-chloro-3-[(3,3-difluoropyrrolidin-1- yl)sulfonyl]-1H-indole-2-carboxamide | Cl | 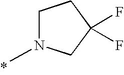 | C(O)NH₂ | 364.8 |
| 40 | 5-chloro-3-{[2- (trifluoromethyl)pyrrolidin-1- yl]sulfonyl}-1H-indole-2-carboxamide | Cl | 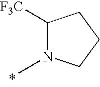 | C(O)NH₂ | 396.8 |
| 41 | 5-chloro-3-[(3-fluoropiperidin-1- yl)sulfonyl]-1H-indole-2-carboxamide | Cl | 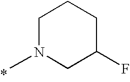 | C(O)NH₂ | 360.8 |
| 42 | 5-bromo-3-[(3,3-difluoropyrrolidin-1- yl)sulfonyl]-1H-indole-2-carboxamide | Br | 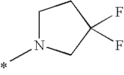 | C(O)NH₂ | 409.2 |
| 43 | 5-bromo-3-[(4,4-difluoropiperidin-1- yl)sulfonyl]-1H-indole-2-carboxamide | Br | 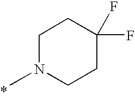 | C(O)NH₂ | 423.3 |
| 44 | 5-bromo-3- [(cyclobutylamino)sulfonyl]-1H-indole- 2-carboxamide | Br | 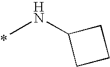 | C(O)NH₂ | 373.2 |
| 45 | 5-bromo-3-[(4-fluoropiperidin-1- yl)sulfonyl]-1H-indole-2-carboxamide | Br | 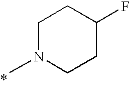 | C(O)NH₂ | 405.3 |
| 46 | 5-chloro-3-[(4-fluoropiperidin-1- yl)sulfonyl]-1H-indole-2-carboxamide | Cl | 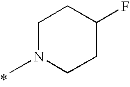 | C(O)NH₂ | 360.8 |

### Example 47

### 5-Bromo-3-(pyrrolidin-1-ylsulfonyl)-N-(1,3-thiazol-2-ylmethyl)-1H-indole-2-carboxamide

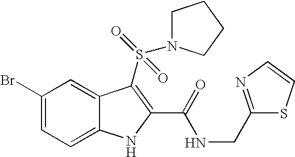

### Step 1: Ethyl 5-bromo-3-(pyrrolidin-1-ylsulfonyl)-1H-indole-2-carboxylate

Pyrrolidine (1820 μL, 21.0 mmol) was added to a solution of ethyl 5-bromo-3-(chlorosulfonyl)-1-(phenylsulfonyl)-1H-indole-2-carboxylate (3.57 g, 7.0 mmol) and pyridine (1400 uL, 14 mmol) in DCM (50 mL) at 0° C. with stirring. The resultant mixture solution was stirred from 0° C. to room temperature for 16 hours. After this time, the solution was diluted with DCM (50 mL) and washed with 1N HCl (3×50 mL),brine (50 mL), dried over Na₂SO₄, filtered, and concentrated. The concentrated residue was purified by LCMS to give the title product as a slightly yellow solid. Analytical LCMS: single peak (214 nm), 3.273 min, ES MS (M+1)=401.

### Step 2: 5-Bromo-3-(pyrrolidin-1-ylsulfonyl)-1H-indole-2-carboxylic acid (I-3)

A mixture of ethyl 5-bromo-3-(pyrrolidin-1-ylsulfonyl)-1H-indole-2-carboxylate (1.61 g, 4.0 mmol) and LiOH (500 mg) in THF/MeOH/H₂O (2:2:1, 50 mL) was heated at 70° C. for 4 hours. After this time, the solution was concentrated to a small volume and then treated with 1N HCl to adjust the solution pH to about 2. The slightly yellow precipitate was collected by filtration and washed with water (3×10 mL). After drying, analytical LCMS confirmed that this yellow solid was the title product. Analytical LCMS: single peak (214 nm), 2.937 min, ES MS (M+1)=373.

### Step 3: 5-Bromo-3-(pyrrolidin-1-ylsulfonyl)-N-(1,3-thiazol-2-ylmethyl)-1H-indole-2-carboxamide

A mixture of 5-bromo-3-(pyrrolidin-1-ylsulfonyl)-1H-indole-2-carboxylic acid (37 mg, 0.1 mmol), PS-DCC (170 mg, 0.20 mmol) and HOBt (14 mg, 0.1 mmol), (1,3-thiazol-2-ylmethyl)amine (dihydrochloride salt, 39 mg, 0.2 mmol), and DIEA (100 uL) in THF/DCM (1:1, 2 mL) was shaken for 16 hours at room temperature. After this time, the resin was filtered and washed with DCM/MeOH (1:1, 4×1.5 mL). The combined organic solution was concentrated and the residue was purified by LCMS to give the title product (TFA salt) as slightly yellow solid Analytical LCMS: single peak (214 nm), 3.254 min, ES MS (M+1)=469; ¹H NMR (500 MHz, d₆-DMSO) δ 13.02 (br, 1H), 9.61 (t, J=6.1 Hz, 1H), 8.12 (d, J=1.8 Hz, 1H), 7.77 (d, J=3.2 Hz, 1H), 7.70 (d, J=3.2 Hz, 1H), 7.53 (d, J=8.7 Hz, 1H), 7.48 (dd, J=8.7, 1.8 Hz, 1H), 4.88 (d, J=6.1 Hz, 1H), 3.16-3.12 (m, 4H), 1.67-1.63 (m, 4H); HRMS, calc'd for C₁₇H₁₈BrN₄O₃S₂ (M+H), 468.9998. found 469.0015.

### Examples 48-65

The compounds in the following table were prepared in accordance with the procedures set forth in Example 47, or routine variations thereof, using the appropriate amine in place of (1,3-thiazol-2-ylmethyl)amine. When the compound was prepared as a salt, the identity of the salt is included in parentheses following the compound name for the free base.

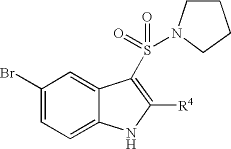

| | | | ES MS |
|---|---|---|---|
| Ex. | Name | R⁴ | (M + 1) |
| 48 | 5-bromo-N-(2-chloro-6- fluorobenzyl)-3-(pyrrolidin-1- ylsulfonyl)-1H-indole-2-carboxamide | 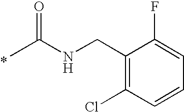 | 515.8 |
| 49 | 5-bromo-N-[2-(1H-imidazol-5- yl)ethyl]-3-(pyrrolidin-1-ylsulfonyl)- 1H-indole-2-carboxamide (TFA salt) | 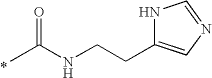 | 467.4 |
| 50 | 5-bromo-N-(pyridin-3-ylmethyl)-3- (pyrrolidin-1-ylsulfonyl)-1H-indole-2- carboxamide (TFA salt) | 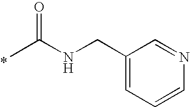 | 464.4 |
| 51 | 5-bromo-N-(2-hydroxybenzyl)-3- (pyrrolidin-1-ylsulfonyl)-1H-indole-2- carboxamide | 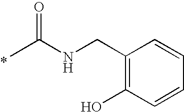 | 479.4 |
| 52 | 5-bromo-N-[3-(1H-imidazol-1- yl)propyl]-3-(pyrrolidin-1-ylsulfonyl)- 1H-indole-2-carboxamide (TFA salt) | 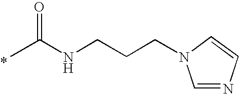 | 481.4 |
| 53 | 5-bromo-N-[2- (difluoromethoxy)benzyl]-3- (pyrrolidin-1-ylsulfonyl)-1H-indole-2- carboxamide | 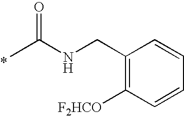 | 529.4 |
| 54 | 5-bromo-N-(pyridin-2-ylmethyl)-3- (pyrrolidin-1-ylsulfonyl)-1H-indole-2- carboxamide (TFA salt) | 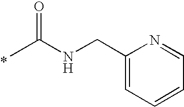 | 464.4 |
| 55 | N-[4-(aminosulfonyl)benzyl]-5- bromo-3-(pyrrolidin-1-ylsulfonyl)- 1H-indole-2-carboxamide | 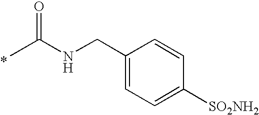 | 542.4 |
| 56 | 5-bromo-N-(2-methoxyethyl)-3- (pyrrolidin-1-ylsulfonyl)-1H-indole-2- carboxamide | C(O)N(H)CH₂CH₂OMe | 431.3 |
| 57 | 5-bromo-3-(pyrrolidin-1-ylsulfonyl)- N-(1,3-thiazol-4-ylmethyl)-1H-indole- 2-carboxamide (TFA salt) | 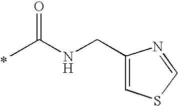 | 470.4 |
| 58 | 5-bromo-N-(isoxazol-3-ylmethyl)-3- (pyrrolidin-1-ylsulfonyl)-1H-indole-2- carboxamide (TFA salt) | 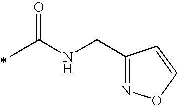 | 454.3 |
| 59 | 5-bromo-N-(1H-pyrazol-5-ylmethyl)- 3-(pyrrolidin-1-ylsulfonyl)-1H-indole- 2-carboxamide (TFA salt) | 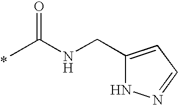 | 453.3 |
| 60 | 5-bromo-N-[(1-methylpyrrolidin-3- yl)methyl]-3-(pyrrolidin-1- ylsulfonyl)-1H-indole-2-carboxamide (TFA salt) | 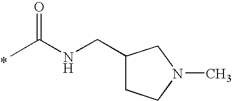 | 470.4 |
| 61 | 5-bromo-N-(1,3-oxazol-4-ylmethyl)- 3-(pyrrolidin-1-ylsulfonyl)-1H-indole- 2-carboxamide (TFA salt) | 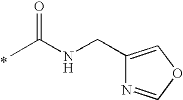 | 454.3 |
| 62 | 5-bromo-N-[(5-phenyl-1H-imidazol- 2-yl)methyl]-3-(pyrrolidin-1- ylsulfonyl)-1H-indole-2-carboxamide (TFA salt) | 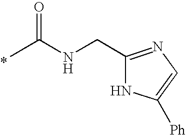 | 529.4 |
| 63 | 5-bromo-N-(3H-imidazo[4,5- b]pyridin-2-ylmethyl)-3-(pyrrolidin-1- ylsulfonyl)-1H-indole-2-carboxamide (TFA salt) | 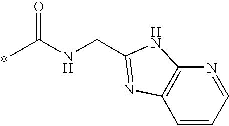 | 504.4 |
| 64 | 5-bromo-N-(pyridin-4-ylmethyl)-3- (pyrrolidin-1-ylsulfonyl)-1H-indole-2- carboxamide (TFA salt) | 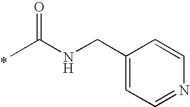 | 464.4 |
| 65 | 5-bromo-3-(pyrrolidin-1-ylsulfonyl)- N-(1,3-thiazol-5-ylmethyl)-1H-indole- 2-carboxamide (TFA salt) | 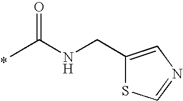 | 470.4 |

### Example 66

### 3-(pyrrolidin-1-ylsulfonyl)-5-vinyl-1H-indole-2-carboxamide

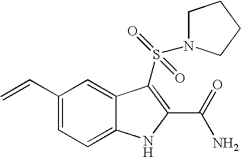

### Step 1: Ethyl 5-bromo-3-(pyrrolidin-1-ylsulfonyl)-1H-indole-2-carboxylate

To a solution of ethyl 5-bromo-3-(chlorosulfonyl)-1-(phenylsulfonyl)-1H-indole-2-carboxylate (5.06 g, 10.0 mmol) in DCM (200 mL) was slowly added a solution of pyrrolidine and DIEA mixture in DCM (50 mL) at 0° C. with stirring. After addition, the resulting mixture was stirred 1 hour at 0° C. and then diluted to 500 mL with DCM and washed with water and brine. The DCM solution was concentrated down and the residue re-dissolved in 2M NH₃-MeOH and heated for 4 hours at 40° C. After this time, the solution was concentrated and the residue was purified by LCMS to give the title product as a slightly yellow solid. Analytical LCMS: single peak (214 nm), 3.273 min, ES MS (M+1)=401.

### Step 2: 5-Bromo-3-(pyrrolidin-1-ylsulfonyl)-1H-indole-2-carboxamide

To a 10 mL microwave tube was charged 200 mg of ethyl 5-bromo-3-(pyrrolidin-1-ylsulfonyl)-1H-indole-2-carboxylate and 2M NH₃-MeOH (5 mL). The tube was heated 100° C. overnight. After cooling to room temperature, the product precipitated from the solution and was collected by filtration. The collected MeOH mother solution was concentrated down, and the residue was purified by LCMS. Analytical LCMS: single peak (214 nm), 3.012 min, ES MS (M+1) 372.

### Step 3: 3-(Pyrrolidin-1-ylsulfonyl)-5-vinyl-1H-indole-2-carboxamide

A mixture of 5-bromo-3-(pyrrolidin-1-ylsulfonyl)-1H-indole-2-carboxamide (22 mg, 0.06 mmol), vinyl boronic acid (0.08 mmol), and a solution of Pd(dppf)Cl₂ (4.3 mg, 0.006 mmol) in THF (1.5 mL) and aqueous Cs₂CO₃ (1M, 1 mL)) was microwaved at 160° C. for 10 minutes. After cooling to room temperature, the reaction mixture was extracted with EtOAc (3×4 mL). The combined organic extracts were washed with water, dried over Na₂SO₄, and concentrated. The concentrated residue was purified by LCMS to afford the title product as a white solid. Analytical LCMS: single peak (214 nm), 2.876 min, ES MS (M+1)=320.1; ¹H NMR (500 MHz, d6-DMSO) δ 12.74 (br, 1H), 8.42 (s, 1H), 8.16 (s, 1H), 7.98 (d, J=1.6 Hz, 1H), 7.56 (dd, J=8.7, 1.6 Hz, 1H), 7.51 (d, J=8.7, Hz, 1H), 6.86 (dd, J=17.7, 10.5 Hz, 1H), 5.77 (d, J=17.7, Hz, 1H), 5.22 (d, J=10.5, Hz, 1H), 3.20-3.15 (m, 4H), 1.67-1.63 (m, 4H); HRMS, calc'd for C₁₅H₁₈N₃O₃S (M+H), 320.01069. found 320.1071.

### Example 67

### 3-(pyrrolidin-1-ylsulfonyl)-5-quinolin-5-yl-1H-indole-2-carboxamide

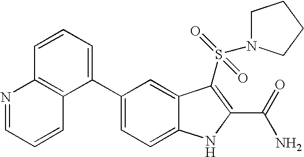

The title compound was prepared in accordance with the procedure set forth in Example 66, wherein 5-quinolin-5-yl boronic acid was employed in place of vinylboronic acid. The title compound was isolated as a TFA salt. ES MS (M+1)=421.5.

### Example 68

### 5-Cyano-3-(pyrrolidin-1-ylsulfonyl)-1H-indole-2-carboxamide

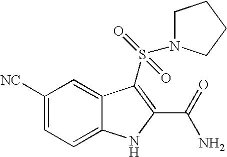

### Step 1: Ethyl 5-bromo-3-(pyrrolidin-1-ylsulfonyl)-1H-indole-2-carboxylate

To a solution of ethyl 5-bromo-3-(chlorosulfonyl)-1-(phenylsulfonyl)-1H-indole-2-carboxylate (5.06 g, 10.0 mmol) in DCM (200 mL) was slowly added a solution of pyrrolidine and DIEA mixture in DCM (50 mL) at 0° C. with stirring. After addition, the resulting mixture was stirred 1 hour at 0° C. and then diluted to 500 mL with DCM and washed with water and brine. The DCM solution was concentrated down and the residue re-dissolved in 2M NH₃-MeOH and heated for 4 hours at 40° C. After this time, the solution was concentrated and the residue was purified by LCMS to give the title product as a slightly yellow solid. Analytical LCMS: single peak (214 nm), 3.273 min, ES MS (M+1)=401.

### Step 2: 5-Bromo-3-(pyrrolidin-1-ylsulfonyl)-1H-indole-2-carboxamide

To a 10 mL microwave tube was charged 200 mg of ethyl 5-bromo-3-(pyrrolidin-1-ylsulfonyl)-1H-indole-2-carboxylate and 2M NH₃-MeOH (5 mL). The tube was heated at 100° C. overnight. After cooling to room temperature, the product precipitated from solution. The product was collected by filtration. The collected MeOH mother solution was concentrated down and the residue was purified by LCMS. Analytical LCMS: single peak (214 nm), 3.012 min, ES MS (M+1) 372.

### Step 3: 5-Cyano-3-(pyrrolidin-1-ylsulfonyl)-1H-indole-2-carboxamide

A mixture of PS—PPh₃ (35 mg, 0.073 mmol), Pd(OAc)₂ (7.5 mg, 0.035 mmol), and de-gassed DMF (3 mL) in a microwave tube was stirred for 2 hours at room temperature under N₂. The tube cap was then removed, and 5-bromo-3-(pyrrolidin-1-ylsulfonyl)-1H-indole-2-carboxamide (150 mg, 0.4 mmol) and Zn(CN)₂ (71 mg, 0.4 mmol) were added to the tube. The tube was re-sealed and degassed 3 times with each time refilling N₂. The reaction mixture was microwaved 1 hour at 140° C. After cooling to room temperature, the resin was filtered and washed with THF (3×3 mL). The combined solution was concentrated and the solid residue was purified by LCMS twice to give the title pure product. Analytical LCMS: single peak (214 nm), 2.506 min, ES MS (M+1)=319.1; ¹H NMR (500 MHz, d6-DMSO)S 13.20 (br, 1H), 8.38 (s, 1H), 8.37 (s, 1H), 8.27 (s, 1H), 7.70 (s, 2H), 3.24-3.16 (m, 4H), 1.70-1.63 (m, 4H); HRMS, calc'd for C₁₄H₁₅N₄O₃S (M+H), 319.0859. found 319.0771.

### Example 69

### Encapsulated Oral Compositions

A capsule formulation suitable for use in the present invention can be prepared by filling standard two-piece gelatin capsules each with 100 mg of the compound of Example 1, 150 mg of lactose, 50 mg of cellulose, and 3 mg of stearic acid. Encapsulated oral compositions containing any one of the compounds of Examples 2 to 68 can be similarly prepared.

### Example 70

### Assay for Inhibition of HIV Reverse Transcriptase

An assay to determine the in vitro inhibition of HIV reverse transcriptase by compounds of the present invention was conducted as follows: HIV-1 RT enzyme (1 nM) was combined with inhibitor or DMSO (10%) in assay buffer (50 mM Tris-HCl, pH 7.8, 1 mM dithiothreitol, 6 mM MgCl₂, 80 mM KCl, 0.025% CHAPS, 0.1 mM EGTA), and the mixture preincubated for 30 minutes at room temperature in microtiter Optiplates (Packard). 100 μL reaction mixtures were initiated with a combination of primer-template substrate (10 nM final concentration) and dNTPs (0.6 μM dNTPs, 0.75 μM [³H]-dGTP). The heterodimeric nucleic acid substrate was generated by annealing the DNA primer pD500 (described in Shaw-Reid et al., J. Biol. Chem., 278: 2777-2780; obtained from Integrated DNA Technologies) to t500, a 500 nucleotide RNA template created by in vitro transcription (see Shaw-Reid et al., J. Biol. Chem., 278: 2777-2780). After 1 hour incubation at 37° C., reactions were quenched by 10 μL streptavidin scintillation proximity assay beads (10 mg/mL, from Amersham Biosciences) in 0.5 M EDTA, pH 8. Microtiter plates were incubated an additional 10 minutes at 37° C. prior to quantification via Topcount (Packard). Representative compounds of the present invention exhibit inhibition of the reverse transcriptase enzyme in this assay. For example, the compounds set forth above in Examples 1 to 68 were tested in the assay and all were found to have IC₅₀ values of less than 1 micromolar.

Analogous assays were conducted substituting mutant HIV strains to determine the in vivo inhibition of compounds of the present invention against mutant HIV reverse transcriptase. In one strain the reverse transcriptase has the Y181C mutation and in the other strain the reverse transcriptase has the K103N mutation. The mutations were generated with the QUIKCHANGE site-directed mutagenesis kit (Stratagene). Certain compounds of the present invention exhibit inhibition of the reverse transcriptase enzyme in these assays. For example, in the Y181C mutant assay the compounds set forth above in Examples 1, 2, 4-6, 10, 13-18, 21, 31, 32, 37-39, 47, 48, 51, 53, 59, 64 and 65 were found to have IC₅₀ values of less than 1 micromolar, and the compounds of Examples 8, 9, 11 and 19 were found to have IC₅₀ values of greater than 1 micromolar and less than 20 micromolar. The compounds of Examples 12 and 68 were tested in the Y181C assay up to 20 micromolar, but specific IC₅₀ values were not obtained; i.e., the IC₅₀ values were greater than 20 micromolar. The compounds set forth in the other Examples were not tested in the Y181C assay. In the K103N mutant assay, the compounds of Examples 10, 47, 48, 51, 53, 59, 64 and 65 were found to have IC₅₀ values of less than 1 micromolar, and the compounds of Examples 1, 6, 9, 13-15, 32, 37, 38 and 68 were found to have ICH, values of greater than 1 micromolar and less than 20 micromolar. The compounds of Examples 2, 4, 5, 8, 11, 12, 16-19, 21 and 31 were tested in the K103N assay up to 20 micromolar, but specific 1050 values were not obtained; i.e., the IC₅₀ values were greater than 20 micromolar. The compounds set forth in the other Examples were not tested in the K103N assay.

### Example 71

### Assay for Inhibition of REV Replication

An assay for the inhibition of acute HIV infection of T-lymphoid cells (alternatively referred to herein as the “spread assay”) was conducted in accordance with Vacca, J. P. et al., Proc. Natl. Acad. Sci. USA 1994, 91: 4096. Representative compounds of the present invention exhibit inhibition of HIV replication in this assay. For example, the compounds set forth in Examples 1-25, 28, 31, 32, 37-46, 48-60 and 63-68 were found to have IC₉₅ values of less than 1 micromolar, and the compounds of Examples 27 and 36 were found to have IC₉₅ values of greater than 1 micromolar and less than 10 micromolar. The compounds of Examples 26, 29, 30, 33-35 and 62 were tested in the spread assay up to 10 micromolar, but specific IC₉₅ values were not obtained; i.e., the IC₉₅ values were greater than 10 micromolar. The compounds of Examples 47 and 61 were not tested.

### Example 72

### Cytotoxicity

Cytotoxicity was determined by microscopic examination of the cells in each well in the spread assay, wherein a trained analyst observed each culture for any of the following morphological changes as compared to the control cultures: pH imbalance, cell abnormality, cytostatic, cytopathic, or crystallization (i.e., the compound is not soluble or forms crystals in the well). The toxicity value assigned to a given compound is the lowest concentration of the compound at which one of the above changes is observed. Representative compounds of the present invention that were tested in the spread assay (see Example 71) were examined for cytotoxicity. For those compounds for which an IC₉₅ value was determined in the spread assay, no cytotoxicity was exhibited at the IC₉₅ concentration; i.e., their toxicity value is greater than their IC₉₅ value. In particular, the compounds set forth in Examples 1-25, 27, 28, 31, 32, 36-46, 48-60 and 63-68 exhibited no cytotoxicity at their IC₉₅ concentrations.

While the foregoing specification teaches the principles of the present invention, with examples provided for the purpose of illustration, the practice of the invention encompasses all of the usual variations, adaptations and/or modifications that come within the scope of the following claims.

1. A method for the treatment of HIV infection, or the treatment of AIDS, wherein the method comprises administering to a subject in need thereof an effective amount of a compound of Formula I, or a pharmaceutically acceptable salt thereof: 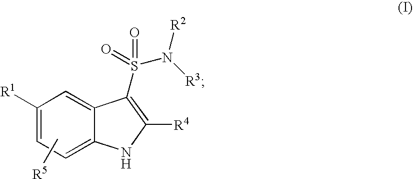
wherein:
R¹ is:
(1) halogen,
(2) CN,
(3) NO₂,
(4) C(O)R^{A},
(5) C(O)OR^{A},
(6) C(O)N(R^{A})R^{B},
(7) SR^{A},
(8) S(O)R^{A},
(9) S(O)₂R^{A},
(10) S(O)₂N(R^{A})R^{B},
(11) N(R^{A})R^{B},
(12) N(R^{A})S(O)₂R^{B},
(13) N(R^{A})C(O)R^{B},
(14) N(R^{A})C(O)ORB,
(15) N(R^{A})S(O)₂N(R^{A})R^{B},
(16) OC(O)N(R^{A})R^{B},
(17) N(R^{A})C(O)N(R^{A})R^{B},
(18) C₁₋₆ alkyl,
(19) C₁₋₆ haloalkyl,
(20) C₂₋₆ alkenyl,
(21) C₂₋₆ allynyl,
(22) OH,
(23) O—C₁₋₆ alkyl,
(24) O—C₁₋₆ haloalkyl,
(25) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(26) CycA,
(27) AryA,
(28) HetA,
(29) HetR,
(30) C₁₋₆ alkyl substituted with CycA, AryA, HetA, or HetR,
(31) J-CycA,
(32) J-AryA,
(33) J-HetA, or
(34) HetR;
J is O, S, S(O), S(O)₂, O—C₁₋₆ alkylene, S—C₁₋₆ alkylene, S(O)—C₁₋₆ alkylene, S(O)₂—C₁₋₆ alkylene, N(R^{A}), N(R^{A})—C₁₋₆ alkylene, C(O), C(O)—C₁₋₆ alkylene-O, C(O)N(R^{A}), C(O)N(R^{A})—C₁₋₆ alkylene, C(O)N(R^{A})—C₁₋₆ alkylene-C(O)O, or C(O)N(R^{A})S(O)₂;
R₂ is:
(1) H,
(2) C₁₋₆ alkyl,
(3) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B}, with the proviso that the OH, O—C₁₋₆ alkyl, or O—C₁₋₆ haloalkyl is not attached to the carbon in C₁₋₆ alkyl that is directly attached to the rest of the molecule,
(4) O—C₁₋₆ alkyl,
(5) CycB,
(6) AryB,
(7) HetB,
(8) HetS, or
(9) C₁₋₆ alkyl substituted with CycB, AryB, HetB, or HetS;
R₃ is:
(1) C₁₋₆ allyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B}, with the proviso that the OH, O—C₁₋₆ alkyl, or O—C₁₋₆ haloalkyl is not attached to the carbon in C₁₋₆ alkyl that is directly attached to the rest of the molecule,
(3) CycB,
(4) AryB,
(5) HetB,
(6) HetS, or
(7) C₁₋₆ alkyl substituted with CycB, AryB, HetB, or HetS;
alternatively R² and R³ together with the N atom to which they are attached form a 4- to 7-membered, saturated or mono-unsaturated heterocyclic ring or a 6- to 10-membered saturated or mono-unsaturated, bridged or fused heterobicyclic ring, wherein the heterocyclic or heterobicyclic ring optionally contains a heteroatom in addition to the nitrogen attached to R² and R³ selected from N, O, and S, wherein the S is optionally oxidized to S(O) or S(O)₂, and wherein the heterocyclic or heterobicyclic ring is optionally substituted with a total of from 1 to 4 substituents, wherein:
(i) from zero to 4 substituents are each independently halogen, CN, C₁₋₆ alkyl, OH, oxo, O—C₁₋₆ alkyl, C₁₋₆ haloalkyl, O—C₁₋₆ haloalkyl, S(O)₂R^{A}, C₁₋₆ alkylene-CN, C₁₋₆ alkylene-OH, or C₁₋₆ alkylene-O—C₁₋₆ alkyl, and
(ii) from zero to 1 substituent is CycB, AryB, HetB, or C₁₋₆ alkyl substituted with CycB, AryB, or HetB;
R⁴ is:
(1) C(O)OH,
(2) C(O)OR^{U},
(3) C(O)NH₂, or
(4) C(O)NR^{V}R^{W};
R⁵ is H or independently has the same definition as R¹;
R^{U} is:
(1) C₁₋₆ alkyl, or
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B};
R^{V} is H or C₁₋₆ alkyl;
R^{W} is:
(1) H,
(2) C₁₋₆ alkyl,
(3) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B}, with the proviso that the OH, O—C₁₋₆ alkyl, or O—C₁₋₆ haloalkyl is not attached to the carbon in C₁₋₆ alkyl that is directly attached to the rest of the molecule,
(4) CycC,
(5) AryC,
(6) HetC,
(7) HetT, or
(8) C₁₋₆ alkyl substituted with CycC, AryC, HetC, or HetT;
CycA is C₃₋₈ cycloalkyl which is optionally substituted with a total of from 1 to 6 substituents, wherein:
(i) from zero to 6 substituents are each independently:
(1) halogen,
(2) CN,
(3) C₁₋₆ alkyl,
(4) OH,
(5) O—C₁₋₆ alkyl, or
(6) C₁₋₆ haloalkyl, and
(ii) from zero to 2 substituents are each independently:
(1) CycD,
(2) AryD,
(3) HetD, or
(4) C₁₋₆ alkyl substituted with AryD, HetD, or CycD;
AryA is aryl which is optionally substituted with a total of from 1 to 6 substituents, wherein:
(i) from zero to 6 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) halogen,
(8) CN,
(9) NO₂,
(10) N(R^{A})R^{B},
(11) C(O)N(R^{A})R^{B},
(12) C(O)R^{A},
(13) C(O)—Cᵢ-6 haloalkyl,
(14) C(O)OR^{A},
(15) OC(O)N(R^{A})R^{B},
(16) SR^{A},
(17) S(O)R^{A},
(18) S(O)₂R^{A},
(19) S(O)₂N(R^{A})R^{B},
(20) N(R^{A})S(O)₂R^{B},
(21) N(R^{A})S(O)₂N(R^{A})R^{B},
(22) N(R^{A})C(O)R^{B},
(23) N(R^{A})C(O)N(R^{A})R^{B},
(24) N(R^{A})C(O)—C(O)N(R^{A})R^{B}, or
(25) N(R^{A})CO₂R^{B}, and
(ii) from zero to 2 substituents are each independently:
(1) CycD,
(2) AryD,
(3) HetD, or
(4) C₁₋₆ alkyl substituted with AryD, HetD, or CycD;
HetA is heteroaryl which is optionally substituted with a total of from 1 to 6 substituents, wherein:
(i) from zero to 6 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) oxo,
(8) halogen,
(9) CN,
(10) NO₂,
(11) N(R^{A})R^{B},
(12) C(O)N(R^{A})R^{B},
(13) C(O)R^{A},
(14) C(O)—C₁₋₆ haloalkyl,
(15) C(O)OR^{A},
(16) OC(O)N(R^{A})R^{B},
(17) SR^{A},
(18) S(O)R^{A},
(19) S(O)₂R^{A},
(20) S(O)₂N(R^{A})R^{B},
(21) N(R^{A})S(O)₂R^{B},
(22) N(R^{A})S(O)₂N(R^{A})R^{B},
(23) N(R^{A})C(O)R^{B},
(24) N(R^{A})C(O)N(R^{A})R^{B},
(25) N(R^{A})C(O)—C(O)N(R^{A})R^{B}, or
(26) N(R^{A})CO₂R^{B}, and
(ii) from zero to 2 substituents are each independently:
(1) CycD,
(2) AryD,
(3) HetD, or
(4) C₁₋₆ alkyl substituted with AryD, HetD, or CycD;
each CycB independently has the same definition as CycA;
each AryB independently has the same definition as AryA;
each HetB independently has the same definition as HetA;
CycC independently has the same definition as CycA;
AryC independently has the same definition as AryA;
HetC independently has the same definition as HetA;
each CycD is independently C₃₋₈ cycloalkyl which is optionally substituted with from 1 to 4 substituents each of which is independently halogen, CN, C₁₋₆ alkyl, OH, O—C₁₋₆ alkyl, C₁₋₆ haloalkyl, C₁₋₆ alkylene-CN, C₁₋₆ alkylene-OH, or C₁₋₆ alkylene-O—C₁₋₆ alkyl;
each AryD is independently phenyl or naphthyl, wherein the phenyl or naphthyl is optionally substituted with from 1 to 5 substituents each of which is independently halogen, CN, NO₂, C₁₋₆ alkyl, C₁₋₆ haloalkyl, OH, O—C₁₋₆ alkyl, O—C₁₋₆ haloalkyl, N(R^{A})R^{B}, C(O)N(R^{A})R^{B}, C(O)R^{A}, C(O)OR^{A}, SR^{A}, S(O)R^{A}, S(O)₂R^{A}, S(O)₂N(R^{A})R^{B}, S(O)₂N(R^{A})C(O)R^{B}, C₁₋₆ alkylene-CN, C₁₋₆ alkylene-NO₂, C₁₋₆ alkylene-OH, C₁₋₆ alkylene-O—C₁₋₆ alkyl, C₁₋₆ alkylene-O—C₁₋₆ haloalkyl, C₁₋₆ alkylene-N(R^{A})R^{B}, C₁₋₆ alkylene-C(O)N(R^{A})R^{B}, C₁₋₆ alkylene-C(O)R^{A}, C₁₋₆ alkylene-C(O)OR^{A}, C₁₋₆ alkylene-SR^{A}, C₁₋₆ alkylene-S(O)R^{A}, C₁₋₆ alkylene-S(O)₂R^{A}, C₁₋₆ alkylene-S(O)₂N(R^{A})R^{B}, or C₁₋₆ alkylene-S(O)₂N(R^{A})C(O)R^{B};
each HetD is independently a 5- or 6-membered heteroaromatic ring containing from 1 to 4 heteroatoms independently selected from N, O and S, wherein each N is optionally in the form of an oxide, and wherein the heteroaromatic ring is optionally substituted with from 1 to 4 substituents each of which is independently halogen, CN, NO₂, C₁₋₆ alkyl, C₁₋₆ haloalkyl, OH, O—C₁₋₆ alkyl, O—C₁₋₆ haloalkyl, N(R^{A})R^{B}, C(O)N(R^{A})R^{B}, C(O)R^{A}, C(O)OR^{A}, SR^{A}, S(O)R^{A}, S(O)₂R^{A}, S(O)₂N(R^{A})R^{B}, S(O)₂N(R^{A})C(O)R^{B}, C₁₋₆ alkylene-CN, C₁₋₆ alkylene-NO₂, C₁₋₆ alkylene-OH, C₁₋₆ alkylene-O—C₁₋₆ alkyl, C₁₋₆ alkylene-O—C₁₋₆ haloalkyl, C₁₋₆ alkylene-N(R^{A})R^{B}, C₁₋₆ alkylene-C(O)N(R^{A})R^{B}, C₁₋₆ alkylene-C(O)R^{A}, C₁₋₆ alkylene-C(O)OR^{A}, C₁₋₆ alkylene-SR^{A}, C₁₋₆ alkylene-S(O)R^{A}, C₁₋₆ alkylene-S(O)₂R^{A}, C₁₋₆ alkylene-S(O)₂N(R^{A})R^{B}, or C₁₋₆ allylene-S(O)₂N(R^{A})C(O)R^{B};
HetR is a 4- to 7-membered, saturated or mono-unsaturated heterocyclic ring containing at least one carbon atom and from 1 to 4 heteroatoms independently selected from N, O and S, where the S is optionally oxidized to S(O) or S(O)₂, and wherein the saturated or mono-unsaturated heterocyclic ring is optionally substituted with from 1 to 4 substituents each of which is independently halogen, CN, C₁₋₆ alkyl, OH, oxo, O—C₁₋₆ alkyl, C₁₋₆ haloalkyl, S(O)₂R^{A}, C₁₋₆ alkylene-CN, C₁₋₆ alkylene-OH, or C₁₋₆ alkylene-O—C₁₋₆ alkyl;
each HetS independently has the same definition as HetR;
HetT independently has the same definition as HetR;
each aryl is independently (i) phenyl, (ii) a 9- or 10-membered bicyclic, fused carbocylic ring system in which at least one ring is aromatic, or (iii) an 11- to 14-membered tricyclic, fused carbocyclic ring system in which at least one ring is aromatic;
each heteroaryl is independently (i) a 5- or 6-membered heteroaromatic ring containing from 1 to 4 heteroatoms independently selected from N, O and S, wherein each N is optionally in the form of an oxide, or (ii) a 9- or 10-membered bicyclic, fused ring system containing from 1 to 4 heteroatoms independently selected from N, O and S, wherein either one or both of the rings contain one or more of the heteroatoms, at least one ring is aromatic, each N is optionally in the form of an oxide, and each S in a ring which is not aromatic is optionally S(O) or S(O)₂;
each R^{A} is independently H or C₁₋₆ alkyl; and
each R^{B} is independently H or C₁₋₆ alkyl.

2. The method according to claim 1, wherein the compound of Formula I, or a pharmaceutically acceptable salt thereof, is as defined in claim 1, with the proviso that when R⁵ is H, then R¹ is:
(1) C(O)R^{A},
(2) C(O)OR^{A},
(3) C(O)N(R^{A})R^{B},
(4) SR^{A},
(5) S(O)R^{A},
(6) S(O)₂R^{A},
(7) S(O)₂N(R^{A})R^{B},
(8) N(C₁₋₆ alkyl)S(O)₂R^{B},
(9) N(C₁₋₆ alkyl)C(O)R^{B},
(10) N(R^{A})C(O)ORB,
(11) N(R^{A})S(O)₂N(R^{A})R^{B},
(12) OC(O)N(R^{A})R^{B},
(13) N(R^{A})C(O)N(R^{A})R^{B},
(14) C₁₋₆ alkyl,
(15) C₁₋₆ haloalkyl,
(16) C₂₋₆ alkenyl,
(17) C₂₋₆ allynyl,
(18) OH,
(19) O—C₁₋₆ alkyl,
(20) O—C₁₋₆ haloalkyl,
(21) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(22) CycA,
(23) AryA,
(24) HetA,
(25) HetR,
(26) C₁₋₆ alkyl substituted with CycB, AryB, HetB, or HetR,
(27) J-CycA,
(28) J-AryA,
(29) J-HetA, or
(30) J-HetR.

3. The method according to claim 1, wherein the compound of Formula I, or a pharmaceutically acceptable salt thereof, is as defined in claim 1, with the proviso that when R⁵ is H, then R¹ is:
(1) C(O)R^{A},
(2) C(O)OR^{A},
(3) C(O)N(R^{A})R^{B},
(4) SR^{A},
(5) S(O)R^{A},
(6) S(O)₂R^{A},
(7) S(O)₂N(R^{A})R^{B},
(8) N(R^{A})C(O)ORB,
(9) N(R^{A})S(O)₂N(R^{A})R^{B},
(10) OC(O)N(R^{A})R^{B},
(11) N(R^{A})C(O)N(R^{A})R^{B},
(12) C₁₋₆ alkyl,
(13) C₁₋₆ haloalkyl,
(14) C₂₋₆ alkenyl,
(15) C₂₋₆ alkynyl,
(16) OH,
(17) O—C₁₋₆ alkyl,
(18) O—C₁₋₆ haloalkyl,
(19) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(20) CycA,
(21) AryA,
(22) HetA,
(23) HetR,
(24) C₁₋₆ alkyl substituted with CycB, AryB, HetB, or HetR,
(25) J-CycA,
(26) AryA,
(27) J-HetA, or
(28) J-HetR.

4. The method according to claim 1, wherein in the compound of Formula I, or a pharmaceutically acceptable salt thereof:
R¹ is:
(1) F, Cl, or Br,
(2) CN,
(3) NO₂,
(4) C(O)—C₁₋₄ alkyl,
(5) C(O)O—C₁₋₄ alkyl,
(6) C(O)N(R^{A})R^{B},
(7) S—C₁₋₄ alkyl,
(8) S(O)—C₁₋₄ alkyl,
(9) S(O)₂—C₁₋₄ alkyl,
(10) S(O)₂N(R^{A})R^{B},
(11) N(R^{A})R^{B},
(12) N(H)S(O)₂—C₁₋₄ alkyl,
(13) N(H)C(O)—C₁₋₄ alkyl,
(14) N(C₁₋₄ allyl)S(O)₂—C₁₋₄ alkyl,
(15) N(C₁₋₄ allyl)C(O)—C₁₋₄ alkyl,
(16) N(H)C(O)O—C₁₋₄ alkyl,
(17) N(C₁₋₄ alkyl)C(O)O—C₁₋₄ alkyl,
(18) N(H)S(O)₂N(R^{A})R^{B},
(19) N(C₁₋₄ allyl)S(O)₂N(R^{A})R^{B},
(20) OC(O)N(R^{A})R^{B},
(21) N(H)C(O)N(R^{A})R^{B},
(22) N(C₁₋₄ alkyl)C(O)N(R^{A})R^{B},
(23) C₁₋₄ alkyl,
(24) C₁₋₄ fluoroalkyl,
(25) C₂₋₄ alkenyl,
(26) C₂₋₄ alkynyl,
(27) OH,
(28) O—C₁₋₄ alkyl,
(29) O—C₁₋₄ fluoroalkyl,
(30) C₁₋₄ alkyl substituted with OH, O—C₁₋₄ alkyl, O—C₁₋₄ fluoroalkyl, CN, NO₂, N(R^{A})R^{B}, C(O)N(R^{A})R^{B}, C(O)—C₁₋₄ alkyl, CO₂—C₁₋₄ alkyl, S—C₁₋₄ alkyl, S(O)—C₁₋₄ alkyl, S(O)₂—C₁₋₄ alkyl, S(O)₂N(R^{A})R^{B}, N(H)C(O)—C₁₋₄ alkyl, N(C₁₋₄ alkyl)^{C}(O)—C₁₋₄ alkyl, N(H)CO₂—C₁₋₄ alkyl, N(C₁₋₄ alkyl)CO₂—C₁₋₄ alkyl, N(H)S(O)₂—C₁₋₄ alkyl, N(C₁₋₄ alkyl)S(O)₂—C₁₋₄ alkyl, N(H)S(O)₂N(R^{A})R^{B}, N(C₁₋₄ alkyl)S(O)₂N(R^{A})R^{B}, OC(O)N(R^{A})R^{B}, N(H)C(O)N(R^{A})R^{B}, or N(C₁₋₄ alkyl)C(O)N(R^{A})R^{B};
(31) CycA,
(32) AryA,
(33) HetA,
(34) HetR, or
(35) C₁₋₄ alkyl substituted with CycA, AryA, HetA, or HetR;
R² is:
(1) C₁₋₄ alkyl,
(2) C₁₋₄ alkyl substituted with OH, O—C₁₋₄ alkyl, O—C₁₋₄ fluoroalkyl, CN, NO₂, N(R^{A})R^{B}, C(O)N(R^{A})R^{B}, C(O)—C₁₋₄ alkyl, CO₂—C₁₋₄ alkyl, S—C₁₋₄ alkyl, S(O)—C₁₋₄ alkyl, S(O)₂—C₁₋₄ alkyl, S(O)₂N(R^{A})R^{B}, N(H)C(O)—C₁₋₄ alkyl, N(C₁₋₄ alkyl)C(O)—C₁₋₄ alkyl, N(H)CO₂—C₁₋₄ alkyl, N(C₁₋₄ alkyl)CO₂—C₁₋₄ alkyl, N(H)S(O)₂—C₁₋₄ alkyl, N(C₁₋₄ alkyl)S(O)₂—C₁₋₄ alkyl, N(H)S(O)₂N(R^{A})R^{B}, N(C₁₋₄ alkyl)S(O)₂N(R^{A})R^{B}, OC(O)N(R^{A})R^{B}, N(H)C(O)N(R^{A})R^{B}, or N(C₁₋₄ alkyl)C(O)N(R^{A})R^{B}, with the proviso that the OH, O—C₁₋₄ alkyl, or O—C₁₋₄ haloalkyl is not attached to the carbon in C₁₋₄ alkyl that is directly attached to the rest of the molecule,
(3) O—C₁₋₄ alkyl,
(4) CycB,
(5) AryB,
(6) HetB,
(7) HetS, or
(8) C₁₋₄ alkyl substituted with CycB, AryB, HetB, or HetS;
R³ is H or C₁₋₄ alkyl;
alternatively R² and R³ together with the N atom to which they are attached form a 4- to 7-membered, saturated or mono-unsaturated heterocyclic ring or a 6- to 10-membered saturated or mono-unsaturated, bridged or fused heterobicyclic ring, wherein the heterocyclic or heterobicyclic ring optionally contains a heteroatom in addition to the nitrogen attached to R² and R³ selected from N, O, and S, wherein the S is optionally oxidized to S(O) or S(O)₂, and wherein the heterocyclic or heterobicyclic ring is optionally substituted with from 1 to 4 substituents each of which is independently Cl, Br, F, CN, C₁₋₄ alkyl, OH, oxo, O—C₁₋₄ alkyl, C₁₋₄ fluoroalkyl, O—C₁₋₄ fluoroalkyl, S(O)₂—C₁₋₄ alkyl, C₁₋₄ alkylene-CN, C₁₋₄ alkylene-OH, or C₁₋₄ alkylene-O—C₁₋₄ alkyl;
R⁴ is:
(1) C(O)O—C₁₋₄ alkyl,
(2) C(O)NH₂, or
(3) C(O)NR^{V}R^{W};
R₅ is H or independently has the same definition as R₁;
R^{V} is H or C₁₋₄ alkyl; and
R^{W} is:
(1) C₁₋₄ alkyl,
(2) C₁₋₄ alkyl substituted with OH, O—C₁₋₄ alkyl, CN, N(R^{A})R^{B}, C(O)N(R^{A})R^{B}, C(O)—C₁₋₄ alkyl, CO₂—C₁₋₄ alkyl, S—C₁₋₄ alkyl, S(O)—C₁₋₄ alkyl, S(O)₂—C₁₋₄ alkyl, S(O)₂N(R^{A})R^{B}, N(H)C(O)—C₁₋₄ alkyl, N(C₁₋₄ alkyl)C(O)—C₁₋₄ alkyl, N(H)S(O)₂—C₁₋₄ alkyl, or N(C₁₋₄ allyl)S(O)₂—C₁₋₄ alkyl, with the proviso that the OH or O—C₁₋₄ alkyl is not attached to the carbon in C₁₋₄ alkyl that is directly attached to the rest of the molecule,
(3) CycC,
(4) AryC,
(5) HetC,
(6) HetT, or
(7) C₁₋₄ alkyl substituted with CycC, AryC, HetC, or HetT;
CycA is C₃₋₆ cycloalkyl which is optionally substituted with a total of from 1 to 4 substituents, wherein:
(i) from zero to 4 substituents are each independently:
(1) Cl, Br, or F,
(2) CN,
(3) C₁₋₄ alkyl,
(4) OH,
(5) O—C₁₋₄ alkyl, or
(6) C₁₋₄ fluoroalkyl, and
(ii) from zero to 1 substituent which is:
(1) CycD,
(2) AryD,
(3) HetD, or
(4) C₁₋₄ alkyl substituted with AryD, HetD, or CycD;
AryA is phenyl or naphthyl, wherein the phenyl or naphthyl is optionally substituted with a total of from 1 to 5 substituents, wherein:
(i) from zero to 5 substituents are each independently:
(1) C₁₋₄ alkyl,
(2) C₁₋₄ alkyl substituted with OH, O—C₁₋₄ alkyl, O—C₁₋₄ fluoroalkyl, CN, NO₂, N(R^{A})R^{B}, C(O)N(R^{A})R^{B}, C(O)—C₁₋₄ alkyl, CO₂—C₁₋₄ alkyl, S—C₁₋₄ alkyl, S(O)—C₁₋₄ alkyl, S(O)₂—C₁₋₄ alkyl, S(O)₂N(R^{A})R^{B}, N(H)C(O)—C₁₋₄ alkyl, N(C₁₋₄ alkyl)C(O)—C₁₋₄ alkyl, N(H)CO₂—C₁₋₄ alkyl, N(C₁₋₄ alkyl)CO₂—C₁₋₄ alkyl, N(H)S(O)₂—C₁₋₄ alkyl, N(C₁₋₄ alkyl)S(O)₂—C₁₋₄ alkyl, N(H)S(O)₂N(R^{A})R^{B}, N(C₁₋₄ allyl)S(O)₂N(R^{A})R^{B}, OC(O)N(R^{A})R^{B}, N(H)C(O)N(R^{A})R^{B}, or N(C₁₋₄ allyl)C(O)N(R^{A})R^{B};
(3) O—C₁₋₄ alkyl,
(4) C₁₋₄ fluoroalkyl,
(5) O—C₁₋₄ fluoroalkyl,
(6) OH,
(7) Cl, Br, or F,
(8) CN,
(9) NO₂,
(10) N(R^{A})R^{B},
(11) C(O)N(R^{A})R^{B},
(12) C(O)—C₁₋₄ alkyl,
(13) C(O)—C₁₋₄ fluoroalkyl,
(14) C(O)O—C₁₋₄ alkyl,
(15) OC(O)N(R^{A})R^{B},
(16) S—C₁₋₄ alkyl,
(17) S(O)—C₁₋₄ alkyl,
(18) S(O)₂—C₁₋₄ alkyl,
(19) S(O)₂N(R^{A})R^{B},
(20) N(H)S(O)₂—C₁₋₄ alkyl,
(21) N(C₁₋₄ alkyl)S(O)₂—C₁₋₄ alkyl,
(22) N(H)C(O)—C₁₋₄ alkyl,
(23) N(C₁₋₄ alkyl)C(O)—C₁₋₄ alkyl,
(24) N(H)CO₂—C₁₋₄ alkyl, or
(25) N(C₁₋₄ allyl)CO₂—C₁₋₄ alkyl, and
(ii) from zero to 1 substituent which is:
(1) CycD,
(2) AryD,
(3) HetD, or
(4) C₁₋₄ alkyl substituted with AryD, HetD, or CycD;
HetA is (i) a 5- or 6-membered heteroaromatic ring containing from 1 to 4 heteroatoms independently selected from N, O and S, wherein each N is optionally in the form of an oxide, or (ii) a 9- or 10-membered bicyclic, fused ring system containing a total of from 1 to 4 heteroatoms independently selected from zero to 4 N atoms, zero to 2 O atoms, and zero to 2 S atoms, wherein either one or both of the rings contain one or more of the heteroatoms, at least one ring is aromatic, each N is optionally in the form of an oxide, and each S in a ring which is not aromatic is optionally S(O) or S(O)₂; wherein the heteroaromatic ring or the bicyclic, fused ring system is optionally substituted with a total of from 1 to 4 substituents, wherein:
(i) from zero to 4 substituents are each independently:
(1) C₁₋₄ alkyl,
(2) C₁₋₄ alkyl substituted with OH, O—C₁₋₄ alkyl, O—C₁₋₄ fluoroalkyl, CN, NO₂, N(R^{A})R^{B}, C(O)N(R^{A})R^{B}, C(O)—C₁₋₄ alkyl, CO₂—C₁₋₄ alkyl, S—C₁₋₄ alkyl, S(O)—C₁₋₄ alkyl, S(O)₂—C₁₋₄ alkyl, S(O)₂N(R^{A})R^{B}, N(H)C(O)—C₁₋₄ alkyl, N(C₁₋₄ allyl)C(O)—C₁₋₄ alkyl, N(H)CO₂—C₁₋₄ alkyl, N(C₁₋₄ allyl)CO₂—C₁₋₄ alkyl, N(H)S(O)₂—C₁₋₄ alkyl, N(C₁₋₄ allyl)S(O)₂—C₁₋₄ alkyl, N(H)S(O)₂N(R^{A})R^{B}, N(C₁₋₄ alkyl)S(O)₂N(R^{A})R^{B}, OC(O)N(R^{A})R^{B}, N(H)C(O)N(R^{A})R^{B}, or N(C₁₋₄ alkyl)C(O)N(R^{A})R^{B};
(3) O—C₁₋₄ alkyl,
(4) C₁₋₄ fluoroalkyl,
(5) O—C₁₋₄ fluoroalkyl,
(6) OH,
(7) oxo,
(8) Cl, Br, or F,
(9) CN,
(10) NO₂,
(11) N(R^{A})R^{B},
(12) C(O)N(R^{A})R^{B},
(13) C(O)—C₁₋₄ alkyl,
(14) C(O)—C₁₋₄ fluoroalkyl,
(15) C(O)O—C₁₋₄ alkyl,
(16) OC(O)N(R^{A})R^{B},
(17) S—C₁₋₄ alkyl,
(18) S(O)—C₁₋₄
(19) S(O)₂—C₁₋₄ alkyl,
(20) S(O)₂N(R^{A})R^{B},
(21) N(H)S(O)₂—C₁₋₄ alkyl,
(22) N(C₁₋₄ alkyl)S(O)₂—C₁₋₄ alkyl,
(23) N(H)C(O)—C₁₋₄ alkyl,
(24) N(C₁₋₄ alkyl)C(O)—C₁₋₄ alkyl,
(25) N(H)CO₂—C₁₋₄ alkyl, or
(26) N(C₁₋₄ alkyl)CO₂—C₁₋₄ alkyl, and
(ii) from zero to 1 substituent which is:
(1) CycD,
(2) AryD,
(3) HetD, or
(4) C₁₋₄ alkyl substituted with AryD, HetD, or CycD;
CycB independently has the same definition as CycA;
AryB independently has the same definition as AryA;
HetB independently has the same definition as HetA;
CycC independently has the same definition as CycA;
AryC independently has the same definition as AryA;
HetC independently has the same definition as HetA;
each CycD is independently C₃₋₆ cycloalkyl which is optionally substituted with from 1 to 4 substituents each of which is independently Cl, Br, F, C₁₋₄ alkyl, OH, O—C₁₋₄ alkyl, C₁₋₄ fluoroalkyl, C₁₋₄ alkylene-OH, or C₁₋₄ alkylene-O—C₁₋₄ alkyl;
each AryD is independently phenyl, wherein the phenyl is optionally substituted with from 1 to 5 substituents each of which is independently Cl, Br, F, CN, NO₂, C₁₋₄ alkyl, C₁₋₄ fluoroalkyl, OH, O—C₁₋₄ alkyl, O—C₁₋₄ fluoroalkyl, N(R^{A})R^{B}, C(O)N(R^{A})R^{B}, C(O)—C₁₋₄ alkyl, C(O)O—C₁₋₄ alkyl, S—C₁₋₄ alkyl, S(O)—C₁₋₄ alkyl, S(O)₂—C₁₋₄ alkyl, S(O)₂N(R^{A})R^{B}, S(O)₂N(R^{A})C(O)—C₁₋₄ alkyl, C₁₋₄ alkylene-OH, C₁₋₄ alkylene-O—C₁₋₄ alkyl, C₁₋₄ alkylene-N(R^{A})R^{B}, C₁₋₄ alkylene-C(O)N(R^{A})R^{B}, or C₁₋₄ alkylene-S(O)₂N(R^{A})R^{B};
each HetD is independently a 5- or 6-membered heteroaromatic ring containing from 1 to 4 heteroatoms independently selected from N, O and S, wherein each N is optionally in the form of an oxide, and wherein the heteroaromatic ring is optionally substituted with from 1 to 4 substituents each of which is independently Cl, Br, F, CN, NO₂, C₁₋₄ alkyl, C₁₋₄ fluoroalkyl, OH, O—C₁₋₄ alkyl, or O—C₁₋₄ fluoroalkyl;
each HetR independently is a 4- to 7-membered, saturated or mono-unsaturated heterocyclic ring containing at least one carbon atom and from 1 to 4 heteroatoms independently selected from N, O and S, where the S is optionally oxidized to S(O) or S(O)₂, and wherein the saturated or mono-unsaturated heterocyclic ring is optionally substituted with from 1 to 4 substituents each of which is independently Cl, Br, F, C₁₋₄ alkyl, oxo, O—C₁₋₄ alkyl, C₁₋₄ fluoroalkyl, S(O)₂—C₁₋₄ alkyl, or C₁₋₄ alkylene-O—C₁₋₄ alkyl;
HetS independently has the same definition as HetR; and
HetT independently has the same definition as HetR;
each R^{A} is independently H or C₁₋₄ alkyl; and
each R^{B} is independently H or C₁₋₄ alkyl.

5. The method according to claim 4, wherein the compound of Formula I, or a pharmaceutically acceptable salt thereof, is as defined in claim 4, with the proviso that when R⁵ is H, then R¹ is:
(1) C(O)—C₁₋₄ alkyl,
(2) C(O)O—C₁₋₄ alkyl,
(3) C(O)N(R^{A})R^{B},
(4) S—C₁₋₄ alkyl,
(5) S(O)—C₁₋₄ alkyl,
(6) S(O)₂—C₁₋₄ alkyl,
(7) S(O)₂N(R^{A})R^{B},
(8) N(C₁₋₄ alkyl)S(O)₂—C₁₋₄ alkyl,
(9) N(C₁₋₄ alkyl)C(O)—C₁₋₄ alkyl,
(10) N(H)C(O)O—C₁₋₄ alkyl,
(11) N(C₁₋₄ alkyl)C(O)O—C₁₋₄ alkyl,
(12) N(H)S(O)₂N(R^{A})R^{B},
(13) N(C₁₋₄ alkyl)S(O)₂N(R^{A})R¹³,
(14) OC(O)N(R^{A})R^{B},
(15) N(H)C(O)N(R^{A})R^{B},
(16) N(C₁₋₄ alkyl)C(O)N(R^{A})R^{B},
(17) C₁₋₄ alkyl,
(18) C₁₋₄ fluoroalkyl,
(19) C₂₋₄ alkenyl,
(20) C₂₋₄ alkynyl,
(21) OH,
(22) O—C₁₋₄ alkyl,
(23) O—C₁₋₄ fluoroalkyl,
(24) C₁₋₄ alkyl substituted with OH, O—C₁₋₄ alkyl, O—C₁₋₄ fluoroalkyl, CN, NO₂, N(R^{A})R^{B}, C(O)N(R^{A})R^{B}, C(O)—C₁₋₄ alkyl, CO₂—C₁₋₄ alkyl, S—C₁₋₄ alkyl, S(O)—C₁₋₄ alkyl, S(O)₂—C₁₋₄ alkyl, S(O)₂N(R^{A})R^{B}, N(H)C(O)—C₁₋₄ alkyl, N(C₁₋₄ alkyl)C(O)—C₁₋₄ alkyl, N(H)CO₂—C₁₋₄ alkyl, N(C₁₋₄ alkyl)CO₂—C₁₋₄ alkyl, N(H)S(O)₂—C₁₋₄ alkyl, N(C₁₋₄ alkyl)S(O)₂—C₁₋₄ alkyl, N(H)S(O)₂N(R^{A})R^{B}, N(C₁₋₄ alkyl)S(O)₂N(R^{A})R^{B}, OC(O)N(R^{A})R^{B}, N(H)C(O)N(R^{A})R^{B}, or N(C₁₋₄ alkyl)C(O)N(R^{A})R^{B};
(25) CycA,
(26) AryA,
(27) HetA,
(28) HetR, or
(29) C₁₋₄ alkyl substituted with CycA, AryA, HetA, or HetR.

6. The method according to claim 4, wherein in the compound of Formula I, or a pharmaceutically acceptable salt thereof:
R¹ is:
(1) Cl,
(2) Br,
(3) CN,
(4) C(O)CH₃,
(5) C(O)OCH₃,
(6) C(O)NH₂,
(9) S(O)₂CH₃,
(10) S(O)₂NH₂,
(11) NH₂,
(12) N(H)S(O)₂CH₃,
(13) N(H)C(O)CH₃,
(14) N(CH₃)S(O)₂CH₃,
(15) N(CH₃)C(O)CH₃,
(16) N(H)C(O)OCH₃,
(17) N(CH₃)C(O)OCH₃,
(18) N(H)S(O)₂NH₂,
(19) N(CH₃)S(O)₂NH₂,
(20) CH₃,
(21) CF₃,
(22) CH═CH₂,
(23) OCH₃,
(24) OCF₃,
(25) CycA,
(26) AryA,
(27) HetA,
(28) (CH₂)₁₋₃-CycA,
(29) (CH₂)₁₋₃-AryA, or
(30) (CH₂)₁₋₃-HetA;
R² is:
(1) C₁₋₃ alkyl,
(2) (CH₂)₂₋₃OH,
(3) (CH₂)₂₋₃OCH₃,
(4) (CH₂)₂₋₃ CF₃,
(5) C₁₋₃ alkyl substituted with CN, NH₂, NH(CH₃), N(CH₃)₂, C(O)NH₂, C(O)NH(CH₃), C(O)N(CH₃)₂, C(O)CH₃, CO₂CH₃, SCH₃, S(O)CH₃, S(O)₂CH₃, S(O)₂NH₂, S(O)₂NH(CH₃), S(O)₂N(CH₃)₂, N(H)C(O)CH₃, or N(CH₃)C(O)CH₃,
(6) O—C₁₋₃ alkyl,
(7) C₃₋₆ cycloalkyl,
(8) HetS, or
(9) (CH₂)₁₋₃-HetS;
R³ is H or CH₃;
alternatively R² and R³ together with the N atom to which they are attached form a saturated or mono-unsaturated heterocyclic ring selected from the group consisting of: 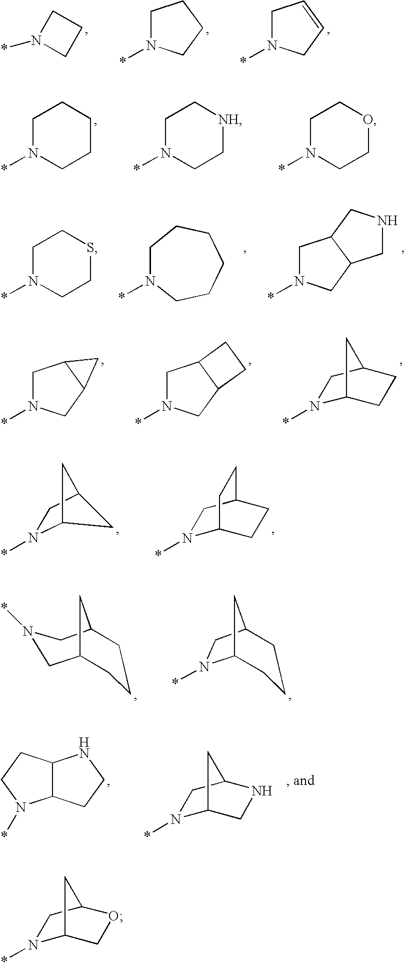
wherein the asterisk denotes the point of attachment of the heterocyclic ring to the rest of the molecule, and wherein the saturated or mono-unsaturated heterocyclic ring is optionally substituted with from 1 to 4 substituents each of which is independently Cl, Br, F, CN, CH₃, oxo, SO₂CH₃, OCH₃, CF₃, or CH₂OCH₃;
R⁴ is:
(1) C(O)OC_{—}3 alkyl,
(2) C(O)NH₂, or
(3) C(O)NR^{V}R^{W};
R^{V} is H or CH₃;
R^{W} is:
(1) C₁₋₃ alkyl,
(2) (CH₂)₂₋₃OH,
(3) (CH₂)₂₋₃OCH₃,
(3) (CH₂)₂₋₃OCF₃,
(4) C₁₋₃ alkyl substituted with CN, N112, NH(CH₃), N(CH₃)₂, C(O)NH₂, C(O)NH(CH₃), C(O)N(CH₃)₂, C(O)CH₃, CO₂CH₃, SCH₃, S(O)CH₃, S(O)₂CH₃, S(O)₂NH₂, S(O)₂NH(CH₃), S(O)₂N(CH₃)₂, N(H)C(O)CH₃, or N(CH₃)C(O)CH₃,
(5) CycC,
(6) AryC,
(7) HetC,
(8) HetT, or
(9) (CH₂)₁₋₃-CycC, (CH₂)₁₋₃-ArYC, (CH₂)₁₋₃-HetC, or (CH₂)₁₋₃-HetT;
R⁵ is H;
CycA is C₃₋₆ cycloalkyl;
AryA is phenyl which is optionally substituted with from 1 to 3 substituents each of which is independently Cl, Br, F, CH₃, OCH₃, CF₃, OCF₃, OCHF₂, OCH₂F, OH, SO₂CH₃, SO₂NH₂, C(O)NH(CH₃), or C(O)N(CH₃)₂;
HetA is a 5- or 6-membered heteroaromatic ring selected from the group consisting of pyrrolyl, thienyl, furanyl, pyrazolyl, imidazolyl, triazolyl, tetrazolyl, oxazolyl, thiazolyl, isoxazolyl, isothiazolyl, oxadiazolyl, pyridinyl, pyrazinyl, and pyrimidinyl, wherein the heteroaromatic ring is optionally substituted with a total of from 1 to 3 substituents, each of which is independently Cl, Br, F, CH₃, or OCH₃;
CycC independently has the same definition as CycA;
AryC independently has the same definition as AryA;
HetC is (i) a 5- or 6-membered heteroaromatic ring selected from the group consisting of pyrrolyl, thienyl, furanyl, pyrazolyl, imidazolyl, triazolyl, tetrazolyl, oxazolyl, thiazolyl, isoxazolyl, isothiazolyl, oxadiazolyl, pyridinyl, pyrazinyl, and pyrimidinyl or (ii) a bicyclic, fused ring system selected from the group consisting of 2,3-dihydrobenzo-1,4-dioxinyl, benzo-1,3-dioxolyl, quinolinyl, isoquinolinyl, quinazolinyl, naphthyridinyl, benzoxazinyl, cinnolinyl, and 4H-imidazo[4,5-b]pyridinyl; wherein the heteroaromatic ring or the bicyclic, fused ring system is optionally substituted with a total of from 1 to 3 substituents, wherein from zero to 3 substituents are each independently Cl, Br, F, CH₃, or OCH₃, and from zero to 1 substituent is phenyl;
HetS is a saturated heterocyclic ring selected from the group consisting of pyrrolidinyl, tetrahydrofuranyl, piperidinyl, piperazinyl, morpholinyl, and thiomorpholinyl, wherein the saturated heterocyclic ring is optionally substituted with from 1 to 4 substituents each of which is independently Cl, Br, F, CH₃, oxo, OCH₃, CF₃, SO₂CH₃, or CH₂OCH₃; and
HetT independently has the same definition as HetS.

7. The method according to claim 6, wherein in the compound of Formula I, or a pharmaceutically acceptable salt thereof:
R¹ is Cl or Br;
R² is:
(1) C₁₋₃ alkyl,
(2) (CH₂)₂₋₃OH,
(3) (CH₂)₂₋₃OCH₃,
(4) (CH₂)₁₋₂NH₂, (CH₂)₁₋₂C(O)NH₂, or (CH₂)₁₋₂S(O)₂NH₂,
(5) OCH₃,
(6) C₃₋₆ cycloalkyl, or
(7) CH₂-HetS;
R³ is H or CH₃;
alternatively R² and R³ together with the N atom to which they are attached form a saturated or mono-unsaturated heterocyclic ring selected from the group consisting of: 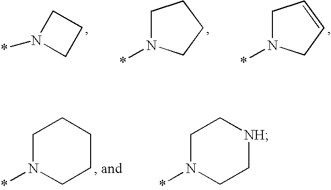
wherein the saturated or mono-unsaturated heterocyclic ring is optionally substituted with from 1 to 4 substituents each of which is independently Cl, Br, F, CN, CH₃, oxo, OCH₃, CF₃, or CH₂OCH₃; and
R⁴ is C(O)OCH₃, C(O)OCH₂CH₃, C(O)NH₂, C(O)N(H)CH₂CH₂OH, C(O)N(H)CH₂CH₂OCH₃, C(O)N(H)(CH₂)₁₋₃-ArYC, C(O)N(H)(CH₂)₁₋₃-HetC, or C(O)N(H)(CH₂)₁₋₃-HetT.

8. The method according to claim 1, wherein the compound of Formula I, or a pharmaceutically acceptable salt thereof, is selected from the group consisting of:
5-bromo-3-(pyrrolidin-1-ylsulfonyl)-1H-indole-2-carboxamide;
5-chloro-3-[(cyclohexylamino)sulfonyl]-1H-indole-2-carboxamide;
5-chloro-3-[(cyclobutylamino)sulfonyl]-1H-indole-2-carboxamide;
5-chloro-3-[(cyclopentylamino)sulfonyl]-1H-indole-2-carboxamide;
5-chloro-3-(piperidin-1-ylsulfonyl)-1H-indole-2-carboxamide;
5-chloro-3-(pyrrolidin-1-ylsulfonyl)-1H-indole-2-carboxamide;
3-(azetidin-1-ylsulfonyl)-5-chloro-1H-indole-2-carboxamide;
5-bromo-3-[cyclopropyl(methyl)amino]sulfonyl 1-1H-indole-2-carboxamide;
3-({[2-(aminosulfonyl)ethyl]amino}sulfonyl)-5-bromo-1H-indole-2-carboxamide;
5-bromo-3-(2,5-dihydro-1H-pyrrol-1-ylsulfonyl)-1H-indole-2-carboxamide;
5-bromo-3-[(cyclopropylamino)sulfonyl]-1H-indole-2-carboxamide;
5-bromo-3-{[methoxy(methyl)amino]sulfonyl}-1H-indole-2-carboxamide;
5-bromo-3-(piperidin-1-ylsulfonyl)-1H-indole-2-carboxamide;
5-bromo-3-(pyrrolidin-1-ylsulfonyl)-1H-indole-2-carboxamide;
5-bromo-3-{[(2S)-2-(methoxymethyl)pyrrolidin-1-yl]sulfonyl}-1H-indole-2-carboxamide;
5-bromo-3-[(cyclohexylamino)sulfonyl]-1H-indole-2-carboxamide;
5-bromo-3-[(cyclopentylamino)sulfonyl]-1H-indole-2-carboxamide;
5-bromo-3-{[(tetrahydrofuran-2-ylmethyl)amino]sulfonyl}-1H-indole-2-carboxamide;
5-bromo-3-{[(2S)-2-(trifluoromethyl)pyrrolidin-1-yl]sulfonyl}-1H-indole-2-carboxamide;
5-bromo-3-[(3-methoxypiperidin-1-yl)sulfonyl]-1H-indole-2-carboxamide;
5-bromo-3-[(3,3-difluoropiperidin-1-yl)sulfonyl]-1H-indole-2-carboxamide;
5-bromo-3-[(3-fluoropyrrolidin-1-yl)sulfonyl]-1H-indole-2-carboxamide;
5-bromo-3-[(propylamino)sulfonyl]-1H-indole-2-carboxamide;
methyl 5-bromo-3-(piperidin-1-ylsulfonyl)-1H-indole-2-carboxylate;
methyl 5-bromo-3-(pyrrolidin-1-ylsulfonyl)-1H-indole-2-carboxylate;
methyl 5-bromo-3-[(cyclopentylamino)sulfonyl]-1H-indole-2-carboxylate;
ethyl 5-bromo-3-(piperidin-1-ylsulfonyl)-1H-indole-2-carboxylate;
ethyl 5-bromo-3-{[(2-methoxyethyl)amino]sulfonyl}-1H-indole-2-carboxylate;
ethyl 5-bromo-3-[(cyclopentylamino)sulfonyl]-1H-indole-2-carboxylate;
ethyl 5-bromo-3-{[2-(trifluoromethyl)pyrrolidin-1-yl]sulfonyl}-1H-indole-2-carboxylate;
5-bromo-N-(2-hydroxyethyl)-3-(piperidin-1-ylsulfonyl)-1H-indole-2-carboxamide;
5-bromo-N-(2-hydroxyethyl)-3-(pyrrolidin-1-ylsulfonyl)-1H-indole-2-carboxamide;
5-bromo-3-[(cyclopropylamino)sulfonyl]-N-(2-hydroxyethyl)-1H-indole-2-carboxamide;
5-bromo-3-[(cyclohexylamino)sulfonyl]-N-(2-hydroxyethyl)-1H-indole-2-carboxamide;
5-bromo-3-[(cyclopentylamino)sulfonyl]-N-(2-hydroxyethyl)-1H-indole-2-carboxamide;
5-bromo-N-(2-hydroxyethyl)-3-[(3-oxopiperazin-1-yl)sulfonyl]-1H-indole-2-carboxamide;
5-chloro-3-(2,5-dihydro-1H-pyrrol-1-ylsulfonyl)-1H-indole-2-carboxamide;
5-chloro-3-[(3-fluoropyrrolidin-1-yl)sulfonyl]-1H-indole-2-carboxamide;
5-chloro-3-[(3,3-difluoropyrrolidin-1-yl)sulfonyl]-1H-indole-2-carboxamide;
5-chloro-3-{[2-(trifluoromethyl)pyrrolidin-1-yl]sulfonyl}-1H-indole-2-carboxamide;
5-chloro-3-[(3-fluoropiperidin-1-yl)sulfonyl]-1H-indole-2-carboxamide;
5-bromo-3-[(3,3-difluoropyrrolidin-1-yl)sulfonyl]-1H-indole-2-carboxamide;
5-bromo-3-[(4,4-difluoropiperidin-1-yl)sulfonyl]-1H-indole-2-carboxamide;
5-bromo-3-[(cyclobutylamino)sulfonyl]-1H-indole-2-carboxamide;
5-bromo-3-[(4-fluoropiperidin-1-yl)sulfonyl]-1H-indole-2-carboxamide;
5-chloro-3-[(4-fluoropiperidin-1-yl)sulfonyl]-1H-indole-2-carboxamide;
5-bromo-3-(pyrrolidin-1-ylsulfonyl)-N-(1,3-thiazol-2-ylmethyl)-1H-indole-2-carboxamide;
5-bromo-N-(2-chloro-6-fluorobenzyl)-3-(pyrrolidin-1-ylsulfonyl)-1H-indole-2-carboxamide;
5-bromo-N-[2-(1H-imidazol-5-yl)ethyl]-3-(pyrrolidin-1-ylsulfonyl)-1H-indole-2-carboxamide;
5-bromo-N-(pyridin-3-ylmethyl)-3-(pyrrolidin-1-ylsulfonyl)-1H-indole-2-carboxamide;
5-bromo-N-(2-hydroxybenzyl)-3-(pyrrolidin-1-ylsulfonyl)-1H-indole-2-carboxamide;
5-bromo-N-[3-(1H-imidazol-1-yl)propyl]-3-(pyrrolidin-1-ylsulfonyl)-1H-indole-2-carboxamide;
5-bromo-N-[2-(difluoromethoxy)benzyl]-3-(pyrrolidin-1-ylsulfonyl)-1H-indole-2-carboxamide;
5-bromo-N-(pyridin-2-ylmethyl)-3-(pyrrolidin-1-ylsulfonyl)-1H-indole-2-carboxamide;
N-[4-(aminosulfonyl)benzyl]-5-bromo-3-(pyrrolidin-1-ylsulfonyl)-1H-indole-2-carboxamide;
5-bromo-N-(2-methoxyethyl)-3-(pyrrolidin-1-ylsulfonyl)-1H-indole-2-carboxamide;
5-bromo-3-(pyrrolidin-1-ylsulfonyl)-N-(1,3-thiazol-4-ylmethyl)-1H-indole-2-carboxamide;
5-bromo-N-(isoxazol-3-ylmethyl)-3-(pyrrolidin-1-ylsulfonyl)-1H-indole-2-carboxamide;
5-bromo-N-(1H-pyrazol-5-ylmethyl)-3-(pyrrolidin-1-ylsulfonyl)-1H-indole-2-carboxamide;
5-bromo-N-[(1-methylpyrrolidin-3-yl)methyl]-3-(pyrrolidin-1-ylsulfonyl)-1H-indole-2-carboxamide;
5-bromo-N-(1,3-oxazol-4-ylmethyl)-3-(pyrrolidin-1-ylsulfonyl)-1H-indole-2-carboxamide;
5-bromo-N-[(5-phenyl-1H-imidazol-2-yl)methyl]-3-(pyrrolidin-1-ylsulfonyl)-1H-indole-2-carboxamide;
5-bromo-N-(3H-imidazo[4,5-b]pyridin-2-ylmethyl)-3-(pyrrolidin-1-ylsulfonyl)-1,1-indole-2-carboxamide;
5-bromo-N-(pyridin-4-ylmethyl)-3-(pyrrolidin-1-ylsulfonyl)-1H-indole-2-carboxamide;
5-bromo-3-(pyrrolidin-1-ylsulfonyl)-N-(1,3-thiazol-5-ylmethyl)-1H-indole-2-carboxamide;
3-(pyrrolidin-1-ylsulfonyl)-5-vinyl-1H-indole-2-carboxamide;
3-(pyrrolidin-1-ylsulfonyl)-5-quinolin-5-yl-1H-indole-2-carboxamide; and
5-cyano-3-(pyrrolidin-1-ylsulfonyl)-1H-indole-2-carboxamide.

9. The method according to claim 1, wherein the compound of Formula I, or a pharmaceutically acceptable salt thereof, is administered in a pharmaceutical composition comprising the compound or its salt and a pharmaceutically acceptable carrier.

10. (canceled)

11. (canceled)

12. A compound, or a pharmaceutically acceptable salt thereof, selected from the group consisting of:
5-bromo-3-(pyrrolidin-1-ylsulfonyl)-1H-indole-2-carboxamide;
5-bromo-3-(piperidin-1-ylsulfonyl)-1H-indole-2-carboxamide;
5-bromo-3-(pyrrolidin-1-ylsulfonyl)-1H-indole-2-carboxamide;
5-bromo-3-{[(2S)-2-(methoxymethyl)pyrrolidin-1-yl]sulfonyl}-1H-indole-2-carboxamide;
5-bromo-3-[(cyclohexylamino)sulfonyl]-1H-indole-2-carboxamide;
5-bromo-3-[(cyclopentylamino)sulfonyl]-1H-indole-2-carboxamide;
5-bromo-3-{[(tetrahydrofuran-2-ylmethyl)amino]sulfonyl}-1H-indole-2-carboxamide;
5-bromo-3-{[(2S)-2-(trifluoromethyl)pyrrolidin-1-yl]sulfonyl}-1H-indole-2-carboxamide;
5-bromo-3-[(3-methoxypiperidin-1-yl)sulfonyl]-1H-indole-2-carboxamide;
5-bromo-3-[(3,3-difluoropiperidin-1-yl)sulfonyl]-1H-indole-2-carboxamide;
5-bromo-3-[(3-fluoropyrrolidin-1-yl)sulfonyl]-1H-indole-2-carboxamide;
5-bromo-3-[(propylamino)sulfonyl]-1H-indole-2-carboxamide;
methyl 5-bromo-3-(piperidin-1-ylsulfonyl)-1H-indole-2-carboxylate;
methyl 5-bromo-3-(pyrrolidin-1-ylsulfonyl)-1H-indole-2-carboxylate;
methyl 5-bromo-3-[(cyclopentylamino)sulfonyl]-1H-indole-2-carboxylate;
ethyl 5-bromo-3-(piperidin-1-ylsulfonyl)-1H-indole-2-carboxylate;
ethyl 5-bromo-3-{[(2-methoxyethyl)amino]sulfonyl}-1H-indole-2-carboxylate;
ethyl 5-bromo-3-[(cyclopentylamino)sulfonyl]-1H-indole-2-carboxylate;
ethyl 5-bromo-3-{[2-(trifluoromethyl)pyrrolidin-1-yl]sulfonyl}-1H-indole-2-carboxylate;
5-bromo-N-(2-hydroxyethyl)-3-(piperidin-1-ylsulfonyl)-1H-indole-2-carboxamide;
5-bromo-N-(2-hydroxyethyl)-3-(pyrrolidin-1-ylsulfonyl)-1H-indole-2-carboxamide;
5-bromo-3-[(cyclopropylamino)sulfonyl]-N-(2-hydroxyethyl)-1H-indole-2-carboxamide;
5-bromo-3-[(cyclohexylamino)sulfonyl]-N-(2-hydroxyethyl)-1H-indole-2-carboxamide;
5-bromo-3-[(cyclopentylamino)sulfonyl]-N-(2-hydroxyethyl)-1H-indole-2-carboxamide;
5-bromo-N-(2-hydroxyethyl)-3-[(3-oxopiperazin-1-yl)sulfonyl]-1H-indole-2-carboxamide;
5-chloro-3-(2,5-dihydro-1H-pyrrol-1-ylsulfonyl)-1H-indole-2-carboxamide;
5-chloro-3-[(3-fluoropyrrolidin-1-yl)sulfonyl]-1H-indole-2-carboxamide;
5-chloro-3-[(3,3-difluoropyrrolidin-1-yl)sulfonyl]-1H-indole-2-carboxamide;
5-chloro-3-{[2-(trifluoromethyl)pyrrolidin-1-yl]sulfonyl}-1H-indole-2-carboxamide;
5-chloro-3-[(3-fluoropiperidin-1-yl)sulfonyl]-1H-indole-2-carboxamide;
5-bromo-3-[(3,3-difluoropyrrolidin-1-yl)sulfonyl]-1H-indole-2-carboxamide;
5-bromo-3-[(4,4-difluoropiperidin-1-yl)sulfonyl]-1H-indole-2-carboxamide;
5-bromo-3-[(cyclobutylamino)sulfonyl]-1H-indole-2-carboxamide;
5-bromo-3-[(4-fluoropiperidin-1-yl)sulfonyl]-1H-indole-2-carboxamide;
5-chloro-3-[(4-fluoropiperidin-1-yl)sulfonyl]-1H-indole-2-carboxamide;
5-bromo-3-(pyrrolidin-1-ylsulfonyl)-N-(1,3-thiazol-2-ylmethyl)-1H-indole-2-carboxamide;
5-bromo-N-(2-chloro-6-fluorobenzyl)-3-(pyrrolidin-1-ylsulfonyl)-1H-indole-2-carboxamide;
5-bromo-N-[2-(1H-imidazol-5-yl)ethyl]-3-(pyrrolidin-1-ylsulfonyl)-1H-indole-2-carboxamide;
5-bromo-N-(pyridin-3-ylmethyl)-3-(pyrrolidin-1-ylsulfonyl)-1H-indole-2-carboxamide;
5-bromo-N-(2-hydroxybenzyl)-3-(pyrrolidin-1-ylsulfonyl)-1H-indole-2-carboxamide;
5-bromo-N-[3-(1H-imidazol-1-yl)propyl]-3-(pyrrolidin-1-ylsulfonyl)-1H-indole-2-carboxamide;
5-bromo-N-[2-(difluoromethoxy)benzyl]-3-(pyrrolidin-1-ylsulfonyl)-1H-indole-2-carboxamide;
5-bromo-N-(pyridin-2-ylmethyl)-3-(pyrrolidin-1-ylsulfonyl)-1H-indole-2-carboxamide;
N-[4-(aminosulfonyl)benzyl]-5-bromo-3-(pyrrolidin-1-ylsulfonyl)-1H-indole-2-carboxamide;
5-bromo-N-(2-methoxyethyl)-3-(pyrrolidin-1-ylsulfonyl)-1H-indole-2-carboxamide;
5-bromo-3-(pyrrolidin-1-ylsulfonyl)-N-(1,3-thiazol-4-ylmethyl)-1H-indole-2-carboxamide;
5-bromo-N-(isoxazol-3-ylmethyl)-3-(pyrrolidin-1-ylsulfonyl)-1H-indole-2-carboxamide;
5-bromo-N-(1H-pyrazol-5-ylmethyl)-3-(pyrrolidin-1-ylsulfonyl)-1H-indole-2-carboxamide;
5-bromo-N-[(1-methylpyrrolidin-3-yl)methyl]-3-(pyrrolidin-1-ylsulfonyl)-1H-indole-2-carboxamide;
5-bromo-N-(1,3-oxazol-4-ylmethyl)-3-(pyrrolidin-1-ylsulfonyl)-1H-indole-2-carboxamide;
5-bromo-N-[(5-phenyl-1H-imidazol-2-yl)methyl]-3-(pyrrolidin-1-ylsulfonyl)-1H-indole-2-carboxamide;
5-bromo-N-(3H-imidazo[4,5-b]pyridin-2-ylmethyl)-3-(pyrrolidin-1-ylsulfonyl)-1H-indole-2-carboxamide;
5-bromo-N-(pyridin-4-ylmethyl)-3-(pyrrolidin-1-ylsulfonyl)-1H-indole-2-carboxamide;
5-bromo-3-(pyrrolidin-1-ylsulfonyl)-N-(1,3-thiazol-5-ylmethyl)-1H-indole-2-carboxamide;
3-(pyrrolidin-1-ylsulfonyl)-5-vinyl-1H-indole-2-carboxamide;
3-(pyrrolidin-1-ylsulfonyl)-5-quinolin-5-yl-1H-indole-2-carboxamide; and
5-cyano-3-(pyrrolidin-1-ylsulfonyl)-1H-indole-2-carboxamide.

13. A pharmaceutical composition comprising an effective amount of a compound according to claim 12, or a pharmaceutically acceptable salt thereof, and a pharmaceutically acceptable carrier.

14. A pharmaceutical combination which is (i) a compound according to claim 12, or a pharmaceutically acceptable salt thereof, and (ii) an HIV infection/AIDS antiviral agent selected from the group consisting of HIV protease inhibitors, nucleoside HIV reverse transcriptase inhibitors, and HIV integrase inhibitors; wherein the compound of (i) or its pharmaceutically acceptable salt and the HIV infection/AIDS antiviral agent of (ii) are each employed in an amount that renders the combination effective for the treatment of HIV infection or the treatment of AIDS.
